(19) United States
(12) Patent Application Publication
(10) Pub. No.: US 2016/0256515 A1
(43) Pub. Date: Sep. 8, 2016

(54) COMPOSITIONS AND METHODS FOR PREDICTION AND TREATMENT OF HUMAN CYTOMEGALOVIRUS INFECTIONS

(71) Applicant: THE JOHNS HOPKINS UNIVERSITY, Baltimore, MD (US)

(72) Inventor: Ravit Boger, Baltimore, MD (US)

(73) Assignee: THE JOHNS HOPKINS UNIVERSITY, Baltimore, MD (US)

(21) Appl. No.: 15/026,863

(22) PCT Filed: Oct. 1, 2014
(86) PCT No.: PCT/US2014/058570

§ 371 Date: Apr. 1, 2016

Related U.S. Application Data
(60) Provisional application No. 61/885,057, filed on Oct. 1, 2013.
(60) Provisional application No. 61/970,216, filed on Mar. 25, 2014.

(51) Int. Cl. A61K 38/06; A61K 38/05; C12Q 1/68

(52) U.S. Cl. CPC A61K 38/06; C12Q 1/6883; A61K 38/05; C12Q 2600/156

(57) ABSTRACT

The present invention relates to the field of virology. More specifically, the present invention provides compositions and methods useful for diagnosing and treating human cytomegalovirus. In one embodiment, a method for identifying a subject as susceptible to or likely to develop a human cytomegalovirus infection comprises the steps of (a) obtaining a biological sample from the subject; (b) performing an assay on the sample obtained from the subject to identify a mutation in NOD1 and/or NOD2; and (c) identifying the subject as susceptible to likely to develop human cytomegalovirus infection if the NOD1 and/or NOD2 mutation is identified.

### CROSS-REFERENCE TO RELATED APPLICATIONS

This application claims the benefit of U.S. Provisional Application No. 61/970,216, filed Mar. 25, 2014, and U.S. Provisional Application No. 61/885,057, filed Oct. 1, 2013, each of which are incorporated herein by reference in their entireties.

### FIELD OF THE INVENTION

The present invention relates to the field of virology. More specifically, the present invention provides compositions and methods useful for predicting and treating human cytomegalovirus.

### INCORPORATION-BY-REFERENCE OF MATERIAL SUBMITTED ELECTRONICALLY

This application contains a sequence listing. It has been submitted electronically via EFS-Web as an ASCII text file entitled “P12380-03_ST25.txt.” The sequence listing is 4,096 bytes in size, and was created on Sep. 29, 2014. It is hereby incorporated by reference in its entirety.

### BACKGROUND OF THE INVENTION

Infection with human CMV (HCMV), a member of the herpesvirus family, is common in humans. Seroprevalence rates increase with age, reaching 80-90% in individuals older than 80 years [1]. While infection in the normal host is usually asymptomatic, HCMV is a major pathogen in immunocompromised patients and the congenitally-infected newborns [2-4]. In these cohorts infection can be severe, persistent, recurrent, or resistant to anti-viral therapy.

Despite being a very common pathogen, around 10-15% of individuals remain HCMV negative for life. HCMV seronegativity may reflect lack of exposure to the virus; alternatively, host genetics may contribute to susceptibility to HCMV infection. Indeed, host genetics can influence susceptibility to human infection and cytokine production by the innate immune system [5-8].

Mutations in signaling proteins of the innate immune system have been implicated in the severity of herpesvirus infections [9]. The most studied group of pattern recognition receptors (PRRs) in the setting of HCMV infection has been the Toll-like receptors (TLRs). TLR2 was reported to recognize HCMV and trigger an inflammatory cytokine production [10, 11]. CMV-encoded glycoprotein B (gB) and gH were shown to interact with TLR2 and TLR1 [11]. A single nucleotide polymorphism (SNP) in TLR2 (Arg753Gln) was associated with HCMV disease in a cohort of liver transplant recipients [12].

The role of cytosolic proteins in sensing herpes viruses is gaining significant research interest [13]. The cytoplasmic dsDNA sensor ZBP1 was found important in HCMV-mediated activation of IRF3 and its constitutive overexpression inhibited HCMV replication [14]. Interferon (IFN)-inducible protein, IFI16, inhibited HCMV replication by directly blocking Spl-mediated transcription of HCMV genes UL54 and UL44, involved in viral DNA synthesis [15]. Amongst the nucleotide-binding oligomerization domain and leucine rich repeat containing receptors (NLRs), NLRC5 was involved in interferon (IFN)-dependent anti-HCMV immune responses. Infection of human fibroblasts with HCMV, but not heat-inactivated virus, induced NLRC5 mRNA within 24 h following infection and knockdown of NLRC5 impaired the upregulation of interferon alpha (IFN-α) in response to HCMV [16]. Involvement of other NLRs in innate immune response to HCMV and the interaction between these receptors in different cell compartments has not been well-studied.

NOD1 and NOD2 are the most widely studied members of the NLR family. These cytoplasmic receptors are highly expressed in monocytes, macrophages, and dendritic cells [17, 18]. NOD1 is also expressed in epithelial cells, and NOD2 expression can be induced in these cells by inflammatory signals [19]. Mutations in NOD2 are strongly associated with Crohn's disease, whereas mutations in NOD1 have been associated with asthma and atopic eczema [20-22]. NOD1 recognizes a fragment of peptidoglycan (PGN) containing the dipeptide γ-d-glutamyl-meso-diaminopimelic acid (iE-DAP) produced by Gram-negative and some Gram-positive bacteria. NOD2 recognizes muramyl dipeptide (MDP), present on most types of PGN. Although NOD1 and NOD2 are well-established as intracellular sensors of bacteria [17, 21, 23-28], recent studies showed that RNA viruses can also activate NOD2 [29, 30]. NOD2 activation by Respiratory Syncytial Virus (RSV) resulted in its relocalization to the mitochondria and binding to the mitochondrial antiviral-signaling protein (MAVS), a process that was independent of the NOD2 downstream kinase, RIPK2, and resulted in activation of IRF3 and MAVS [29]. The contribution of NOD1 and NOD2 to herpesvirus infections has not been studied.

### SUMMARY OF THE INVENTION

The present invention is based, at least in part, on the discovery that NOD1 and NOD2 can serve as diagnostic markers and therapeutic targets for human cytomegalovirus (HCMV). As described herein, HCMV is recognized by NOD1 and NOD2, a process leading to initiation of anti-viral immune responses. These results open very important opportunities for diagnostic tests in at-risk populations for HCMV disease and for the development of strategic methods for HCMV inhibition.

Human Cytomegalovirus (HCMV) is a major pathogen in immunocompromised hosts and is the most common congenital infection worldwide. Understanding how HCMV is recognized by the innate immune system can assist in developing prophylactic and treatment strategies against it. We report that infection with HCMV significantly induces NOD2 expression. While glycoprotein B is not required for NOD2 induction, a replication competent virion is necessary. Knockdown of NOD2 leads to enhanced HCMV replication along with decreased levels of anti-viral and anti-inflammatory cytokines. Overexpression of NOD2 or its downstream RIPK2 kinase results in decreased HCMV replication and enhanced cytokine responses. However, overexpression of a mutant NOD2, 3020insC, associated with severe Crohn's disease, results in enhanced HCMV replication and decreased intracellular cytokines. These results show for the first time that NOD2 plays a critical role in replication of a persistent DNA virus and may provide a model for studies of HCMV recognition by the host cell. Our findings also provide an initial insight for the potential influence of HCMV in episodes of colitis in patients with Crohn's disease.

Accordingly, in one aspect, the present invention provides methods and compositions useful for treating human cytomegalovirus. In a specific embodiment, a method for treating human cytomegalovirus (HCMV) in a patient in need thereof comprises administering an effective amount of a NOD1 pathway agonist and/or a NOD2 pathway agonist. In certain embodiments, the agonist is selected from the group consisting of a protein, a small molecule, an antibody, and an aptamer. In a specific embodiment, the agonist is a small molecule. In another specific embodiment, the NOD2 pathway agonist is muramyl dipeptide (MDP). In a further embodiment, the NOD1 pathway agonist is L-Ala-γ-D-Glu-mDAP (Tri-DAP).

In yet another embodiment, the present invention provides a method for treating human cytomegalovirus (HCMV) in a patient in need thereof comprising administering an agent that increases the expression or activity of NOD1 and/or NOD2. In further embodiments, variations in NOD2 predict likelihood of CMV disease and therefore serve as important diagnostic and prognostic markers.

In another aspect, the present invention provides compositions and methods useful for identifying a subject as susceptible or likely to develop a human cytomegalovirus infection. In one embodiment, a method for identifying a subject as susceptible to or likely to develop a human cytomegalovirus infection comprises the steps of (a) obtaining a biological sample from the subject; (b) performing an assay on the sample obtained from the subject to identify a mutation in NOD2; and (c) identifying the subject as susceptible to likely to develop human cytomegalovirus infection if the NOD2 mutation is identified. In a specific embodiment, the assay of step (b) comprises sequencing of a region of the NOD2 gene comprising the mutation. In another specific embodiment, the assay of step (b) comprises the steps of (i) extracting DNA from the biological sample; (ii) contacting the DNA with a primer that specifically hybridizes to the NOD2 gene; (iii) amplifying by polymerase chain reaction (PCR) a region of the NOD2 gene that comprises the mutation; and (iv) sequencing the amplification product to identify the presence of the NOD2 mutation. In other embodiments, the NOD2 mutation comprises one or more of R702W, G908R, L1007fs, and R334W. In particular embodiments, the method further comprises performing an assay on the sample obtained from the subject to identify a mutation in one or more of vimentin, NOD1, OAS2, RIG-I, RIPK2, XIAP, Nemo (IKK gamma), IKK epsilon, IRF3, IRF5, and IRF7. In certain embodiments, the XIAP mutations comprise one or more of E99X, C203Y, G39C, K297T, and W323X. In another specific embodiment, the NOD1 mutation is E266K. In yet another embodiment, the RIPK2 mutation comprises K47A.

In a specific embodiment, the method further comprises the step of administering a treatment modality appropriate for a subject susceptible to or likely to develop human cytomegalovirus infection. In certain embodiments, the treatment modality for human cytomegalovirus infection comprises ganciclovir, valganciclovir, foscarnet, cidofovir, and/or cytomegalovirus immune globulin. In a specific embodiment, the treatment modality comprises administering to the subject a NOD1 pathway agonist and/or a NOD2 pathway agonist.

In another embodiment, a method for treating a subject having a human cytomegalovirus infection comprises the steps of (a) obtaining a biological sample from the subject; (b) performing an assay on the sample obtained from the subject to identify a mutation in NOD1 or NOD2; (c) identifying the subject as susceptible to likely to develop human cytomegalovirus infection if the NOD1 and/or NOD2 mutation is identified; and (d) treating the subject with one or more treatment modalities appropriate for a subject having or likely to develop human cytomegalovirus infection. In a specific embodiment, the assay of step (b) comprises sequencing of a region of the NOD1 or NOD2 gene comprising the mutation. In another embodiment, the assay of step (b) comprises the steps of (i) extracting DNA from the biological sample; (ii) contacting the DNA with primers that specifically hybridize to the NOD1 and NOD2 gene; (iii) amplifying by polymerase chain reaction (PCR) a region of the NOD1 and NOD2 gene that comprises the mutation; and (iv) sequencing the amplification product to identify the presence of the NOD1 and/or NOD2 mutation. In certain embodiments, the NOD2 mutation is 3020insC. In other embodiments, the NOD2 mutation comprises one or more of R702W, G908R, L1007fs, and R334W. In particular embodiments, the method further comprises performing an assay on the sample obtained from the subject to identify a mutation in one or more of vimentin, NOD1, OAS2, RIG-I, RIPK2, XIAP, Nemo (IKK gamma), IKK epsilon, IRF3, IRF5, and IRF7. In certain embodiments, the XIAP mutations comprise one or more of E99X, C203Y, G39C, K297T, and W323X. In another specific embodiment, the NOD1 mutation is E266K. In yet another embodiment, the RIPK2 mutation comprises K47A. In certain embodiments, the treatment modality for human cytomegalovirus infection comprises ganciclovir, valganciclovir, foscarnet, cidofovir, and/or cytomegalovirus immune globulin. In another embodiment, the treatment modality comprises administering to the subject a NOD1 pathway agonist and/or a NOD2 pathway agonist.

### BRIEF DESCRIPTION OF THE FIGURES

FIG. 1: HCMV infection induces NOD2 mRNA and protein in HFFs and U373 cells. A. HFFs were infected with HCMV Towne strain and levels of NOD1, NOD2 and Glyceraldehyde 3-phosphate dehydrogenase (GAPDH) mRNAs were measured by qRT-PCR at indicated time points. B. HFFs were infected with HCMV-TB40 and levels of NOD1, NOD2 and GAPDH mRNAs were measured by qRT-PCR at indicated time points. C. HFFs were infected with a clinical isolate of HCMV and levels of NOD1, NOD2 and GAPDH mRNAs were measured by qRT-PCR at indicated time points. D. HFFs were treated with MDP (10 μg/ml) and levels of NOD1 and NOD2 mRNA were measured as in A, B and C, E. U373 glioma cells were infected with HCMV Towne strain and levels of NOD1, NOD2 and GAPDH mRNAs were measured by qRT-PCR at indicated time points. F. HFFs were infected with HCMV (Towne) at MOI of 1 PFU/cell and levels of NOD2 protein and β-actin were determined 48 and 72 hpi. G. HFFs were infected with HCMV (Towne) strain at MOI of 0.03 or 3 PFU/cell and levels of NOD2 protein and β-actin were determined at 48 hpi. Quantitative data represent mean values (±SD) of triplicate determinations from three independent experiments (*p<0.05, **p<0.01, ***p<0.001, one-way ANOVA test).

FIG. 2: Kinetics of NOD1 and NOD2 transcripts in HCMV-infected cells. A. B. HFFs were infected with HCMV Towne strain or treated with MDP (10 μg/ml) and levels of NOD1, NOD2 and GAPDH mRNAs were measured by RT-PCR (A) and qRT-PCR (B) at indicated time points. The data shown are the average of three experiments±SD (*p<0.05, **p<0.01, ***p<0.001, one-way ANOVA test).

FIG. 3: HCMV-encoded glycoprotein B (HCMV-gB) and UV-inactivated virus cannot induce NOD2. A. Left—HFFs were treated with recombinant HCMV-gB (5 μg/ml) for 24 h and 72 h and levels of NOD1 and NOD2 mRNA were measured by qRT-PCR. Right—HFFs were treated with recombinant HCMV-gB (5 μg/ml or 20 μg/ml) for 24 h and levels of NOD2, ISG15 and viperin mRNA were measured by qRT-PCR. B. HFFs were infected with HCMV Towne or a UV-inactivated HCMV Towne for 2, 4, and 24 h and levels of NOD1 and NOD2 mRNA were measured by qRT-PCR. IL8 levels were measured as control at 4 hpi. Quantitative data represent mean values (±SD) of triplicate determinations from three independent experiments (*p<0.05, **p<0.01, ***p<0.001, one-way ANOVA test).

FIG. 4: Overexpression of NOD2 restricts HCMV replication and induces antiviral and pro-inflammatory cytokines. A. U373 cells were transiently transfected with pcDNA4/HisMax, pcDNA4/EGFP, pcDNA4/HisMax-hNOD2 or pcDNA4/HisMax-hRIPK2 plasmid. 24 h after transfection cells were infected with pp28-luciferase HCMV. Luciferase activity was measured in cell lysates at 72 hpi. B. Cell lysates from 4A were used to determine protein expression of HCMV-immediate early (IE1/IE2), early (UL44), and late (pp65) genes. Levels of NOD2 and RIPK2 proteins were measured to confirm NOD2 overexpression; β-actin served as loading control. Western blot data are representative of three independent experiments. Asterisks (*) denote endogenous NOD2 and RIPK2 proteins. C-D. U373 cells were transiently transfected with pcDNA4/HisMax, hNOD2 or hRIPK2 plasmid. 24 h after transfection cells were infected with HCMV Towne and total RNA was isolated at 72 hpi. Levels of IFN-β and IL8 mRNAs were measured by qRT-PCR in non-infected (mock) and HCMV-infected (HCMV) cells. E. HFFs stably expressing empty vector (HFF-control) or HA-tagged NOD2 (HFF-NOD2) were either untreated or treated with 2 μg/ml doxycycline and expression of NOD2 was determined at 48 h using anti-HA antibody. F. HFFs stably expressing empty vector (control) or NOD2 (HFF-NOD2) were induced with doxycycline for 24 h followed by infection with HCMV Towne. Cells were incubated in doxycycline containing media and cell-free supernatants were collected at 96 hpi. Virus progeny released into the supernatants were quantified after infection of fresh HFFs (second cycle) with equal amount of supernatants from control or NOD2-overexpressing HFFs using luciferase assay at 3 dpi (Y-axis on left, in blue) and by real-time PCR (Y-axis on right, in red) in the supernatants of newly-infected HFFs (FIG. 4F). Viral protein expression was determined in newly-infected HFFs at 3 dpi (FIG. 4 G). H, J. Levels of IFN-β and IL8 mRNAs were determined in HFFs-control or HFF-NOD2 cells using qRT-PCR at 96 hpi. I. Levels of IFN-β secreted into the media from HFF-control and HFF-NOD2 cells (first cycle of infection) were measured at 24 hpi using IFN-β ELISA kit. The data shown are the average of three experiments±SD (*p<0.05, **p<0.01, ***p<0.001, one-way ANOVA test).

FIG. 5: Knockdown of NOD2 results in enhanced HCMV replication in HFFs. A. HFF stably expressing control lentiviral vector (HFF-GIPZ) or a lentiviral vector expressing short-hairpin RNA (shRNA) against NOD2 (HFF-shNOD2) were infected with HCMV at MOI=1, and levels of NOD2 mRNA were measured using qRT-PCR at 72 hpi. B. Cell-free supernatants were collected at 96 hpi from HCMV-infected HFF-GIPZ or HFF-shNOD2 cells and used to infect fresh HFFs (second cycle). Luciferase activity was measured at 72 hpi. C. Cell-free supernatants were collected at 3 dpi from HCMV-infected HFF-GIPZ (control) and HFF-shNOD2 cells and used to perform a yield reduction assay in fresh HFFs. D, E. Levels of IFN-β and IL8 mRNA were measured in cell lysates collected at 72 h from non-infected and HCMV-infected HFF-GIPZ and HFF-shNOD2 cells using qRT-PCR. The data shown are the average of three experiments±SD (*p<0.05, **p<0.01, ***p<0.001, one-way ANOVA test).

FIG. 6: Rescue of NOD2 expression in KD cells restores the restriction of HCMV replication. A, B, C. U373 cells were transfected with pcDNA4/HisMax, pcDNA4/HisMax-hNOD2 or pcDNA4/HisMax-hNOD2 3020insC and infected with HCMV after 24 h. Virus replication (by luciferase activity), NOD2 expression (by western blot) and expression of cytokines (by qRT-PCR) were measured. D. U373-GIPZ or U373-shNOD2 cells were transfected with control plasmid pcDNA4/HisMax (pcDNA4), pcDNA4/HisMax-NOD2 (pcDNA4-NOD2), or pcDNA/HisMax-NOD2 3020insC (NOD2 3020insC) followed by HCMV infection 24 h later. HCMV replication was determined using luciferase assay. Quantitative data represent mean values (±SD) of triplicate determinations from two independent experiments (*p<0.05, **p<0.01, ***p<0.001, one-way ANOVA test).

FIG. 7: NOD2 KD results in decreased activation of the NF-κB and IFN pathways. NOD2 KD HFFs (HFF-shNOD2) and control HFFs (HFF-GIPZ) were infected with HCMV Towne and expression of NF-κB (p65) and IRF3 was measured in cytoplasmic and nuclear extracts at 24 hpi. Representative data from three independent experiments are shown.

FIG. 8: HSV1 and HSV2 do not induce NOD2 mRNA expression. A, B. HFFs were infected with HSV1 KOS/Dlux/oriS (MOI=1) or a clinical isolate of HSV2 (MOI=0.1) and levels of NOD1 were determined by qRT-PCR at indicated time points. NOD2 levels were undetected in HSV1- and HSV2-infected cells. Quantitative data represent mean values (±SD) of triplicate determinations from two independent experiments.

FIG. 9: Knockdown of NOD2 results in enhanced HCMV replication in U373 cells. A. U373 cells stably expressing control lentiviral vector (U373-GIPZ) or a lentiviral vector expressing short-hairpin RNA (shRNA) against NOD2 (U373-shNOD2) were infected with HCMV at MOI=1, and levels of NOD2 mRNA were measured using qRT-PCR at 72 hpi. B. Luciferase activity in cell lysates (measured at 96 hpi), virus DNA replication in supernatants (quantified at 96 hpi) and viral DNA replication (quantified at 48 hpi) were determined in cells from 5A. C, D. IFN-β and IL8 transcripts were measured in non-infected and HCMV-infected U373-GIPZ and U373-shNOD2 cells using qRT-PCR at 72 hpi. The data shown are the average of three experiments±SD (*p<0.05, **p<0.01, ***p<0.001, one-way ANOVA test).

FIG. 10. Effect of NOD1 knockdown (A) and NOD1 overexpression (B) on CMV replication. CMV replication was quantified in human foreskin fibroblasts (HFFs) and U373 (glioma) cells using the pp28-luciferase recombinant Towne strain.

FIG. 11. Effect of triDAP on human CMV (HCMV) replication.

FIG. 12. Pretreatment with triDAP followed by CMV infection results in increased mRNA and protein expression of IFN-β (qPCR results are shown in A and B and ELISA of IFN-β is shown in C).

FIG. 13. NOD1 and NOD2 have independent activities in CMV recognition. Left—NOD1 mRNA expression is maintained in the NOD2 KD cells. Right—treatment with triDAP in NOD2 KD cells can initiate independent IFN-β responses.

FIG. 14. NOD1 localization in non-infected (Mock) and CMV-infected HFFs. M=MDP (NOD2 activator); T=triDAP (NOD1 activator).

FIG. 15. Sequence analysis of NOD1 showing the E266K missense mutation.

FIG. 16. NFkb-alpha actin sequence in XhoI and HindIII site of pC1-6-20.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. Definitions

As used herein, the term “modulate” indicates the ability to control or influence directly or indirectly, and by way of non-limiting examples, can alternatively mean inhibit or stimulate, agonize or antagonize, hinder or promote, and strengthen or weaken. Thus, the terms “NOD2 modulator” and “NOD2 pathway modulator” are used interchangeably herein and refer to an agent that modulates the NOD2 pathway. Modulators may be organic or inorganic, small to large molecular weight individual compounds, mixtures and combinatorial libraries of inhibitors, agonists, antagonists, and biopolymers such as peptides, nucleic acids, or oligonucleotides. A modulator may be a natural product or a naturally-occurring small molecule organic compound. In particular, a modulator may be a carbohydrate; monosaccharide; oligosaccharide; polysaccharide; amino acid; peptide; oligopeptide; polypeptide; protein; receptor; nucleic acid; nucleoside; nucleotide; oligonucleotide; polynucleotide including DNA and DNA fragments, RNA and RNA fragments and the like; lipid; retinoid; steroid; glycopeptides; glycoprotein; proteoglycan and the like; and synthetic analogues or derivatives thereof, including peptidomimetics, small molecule organic compounds and the like, and mixtures thereof. A modulator identified according to the invention is preferably useful in the treatment of a disease disclosed herein.

As used herein, an “antagonist” is a type of modulator and the term refers to an agent that binds a target (e.g., a protein) and can inhibit a one or more functions of the target. For example, an antagonist of a protein can bind the protein and inhibit the binding of a natural or cognate ligand to the protein and/or inhibit signal transduction mediated through the protein.

An “agonist” is a type of modulator and refers to an agent that binds a target and can activate one or more functions of the target. For example, an agonist of a protein can bind the protein and activate the protein in the absence of its natural or cognate ligand.

As used herein, the term “antibody” is used in reference to any immunoglobulin molecule that reacts with a specific antigen. It is intended that the term encompass any immunoglobulin (e.g., IgG, IgM, IgA, IgE, IgD, etc.) obtained from any source (e.g., humans, rodents, non-human primates, caprines, bovines, equines, ovines, etc.). Specific types/examples of antibodies include polyclonal, monoclonal, humanized, chimeric, human, or otherwise-human-suitable antibodies. “Antibodies” also includes any fragment or derivative of any of the herein described antibodies. In specific embodiments, antibodies may be raised against NOD2 and used as NOD2 modulators.

The terms “specifically binds to,” “specific for,” and related grammatical variants refer to that binding which occurs between such paired species as antibody/antigen, enzyme/substrate, receptor/agonist, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins. In some embodiments, specific binding between an antigen and an antibody will have a binding affinity of at least 10⁻⁶ M. In other embodiments, the antigen and antibody will bind with affinities of at least 10⁻⁷ M, 10⁻⁸ M to 10⁻⁹ M, 10⁻¹⁰ M, 10⁻¹¹ M, or 10⁻¹² M.

By “specifically hybridizes” is meant that a probe, primer, or oligonucleotide recognizes and physically interacts (that is, base-pairs) with a substantially complementary nucleic acid (for example, a NOD2 nucleic acid) under high stringency conditions, and does not substantially base pair with other nucleic acids.

Optional” or “optionally” means that the subsequently described event or circumstance can or cannot occur, and that the description includes instances where the event or circumstance occurs and instances where it does not.

As used herein, a “subject” or “patient” means an individual and can include domesticated animals, (e.g., cats, dogs, etc.); livestock (e.g., cattle, horses, pigs, sheep, goats, etc.), laboratory animals (e.g., mouse, rabbit, rat, guinea pig, etc.) and birds. In one aspect, the subject is a mammal such as a primate or a human. In particular, the term also includes mammals diagnosed with a human cytomegalovirus infection (HCMV).

As used herein, the term “effective,” means adequate to accomplish a desired, expected, or intended result. More particularly, a “therapeutically effective amount” as provided herein refers to an amount of a NOD2 pathway modulator of the present invention, either alone or in combination with another therapeutic agent, necessary to provide the desired therapeutic effect, e.g., an amount that is effective to prevent, alleviate, or ameliorate symptoms of disease or prolong the survival of the subject being treated. In a specific embodiment, the term “therapeutically effective amount” as provided herein refers to an amount of a NOD2 pathway modulator, necessary to provide the desired therapeutic effect, e.g., an amount that is effective to prevent, alleviate, or ameliorate symptoms of disease or prolong the survival of the subject being treated. In a particular embodiment, the disease or condition is infection with human cytomegalovirus. As would be appreciated by one of ordinary skill in the art, the exact amount required will vary from subject to subject, depending on age, general condition of the subject, the severity of the condition being treated, the particular compound and/or composition administered, and the like. An appropriate “therapeutically effective amount” in any individual case can be determined by one of ordinary skill in the art by reference to the pertinent texts and literature and/or by using routine experimentation.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease. In a specific embodiment, the disease or condition is infection with HCMV.

The terms “NOD2-related disease, disorder or condition” or “NOD2-mediated disease, disorder or condition,” and the like mean diseases, disorders or conditions associated with aberrant NOD2 activity. In a specific embodiment, the disease or condition is a human cytomegalovirus infection. In general, the term refers to any abnormal state that involves NOD2 activity. The abnormal state can be due, for example, to a genetic defect.

By “high stringency conditions” is meant conditions that allow hybridization comparable with that resulting from the use of a DNA probe of at least 40 nucleotides in length, in a buffer containing 0.5 M NaHPO₄, pH 7.2, 7% SDS, 1 mM EDTA, and 1% BSA (Fraction V), at a temperature of 65° C., or a buffer containing 48% formamide, 4.8×SSC, 0.2 M Tris-Cl, pH 7.6, 1×Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well-known by those skilled in the art of molecular biology. (See, for example, F. Ausubel et al., Current Protocols in Molecular Biology, John Wiley & Sons, New York, N.Y., 1998).

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

Optional” or “optionally” means that the subsequently described event or circumstance can or cannot occur, and that the description includes instances where the event or circumstance occurs and instances where it does not.

The terms “patient,” “individual,” or “subject” are used interchangeably herein, and refer to a mammal, particularly, a human. The patient may have a mild, intermediate or severe disease or condition. The patient may be treatment naïve, responding to any form of treatment, or refractory. The patient may be an individual in need of treatment or in need of diagnosis based on particular symptoms or family history. In some cases, the terms may refer to treatment in experimental animals, in veterinary application, and in the development of animal models for disease, including, but not limited to, rodents including mice, rats, and hamsters; and primates. In particular, the term also includes mammals diagnosed with a NOD2 mediated disease, disorder or condition. By “normal subject” is meant an individual who does not have HCMV as well as an individual who has increased susceptibility for developing HCMV.

“Polypeptide” as used herein refers to any peptide, oligopeptide, polypeptide, gene product, expression product, or protein. A polypeptide is comprised of consecutive amino acids. The term “polypeptide” encompasses naturally occurring or synthetic molecules. In addition, as used herein, the term “polypeptide” refers to amino acids joined to each other by peptide bonds or modified peptide bonds, e.g., peptide isosteres, etc., and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation. See Proteins—Structure and Molecular Properties 2nd Ed., T. E. Creighton, W.H. Freeman and Company, New York (1993); Posttranslational Covalent Modification of Proteins, B. C. Johnson, Ed., Academic Press, New York, pp. 1-12 (1983).

By “probe,” “primer,” or oligonucleotide is meant a single-stranded DNA or RNA molecule of defined sequence that can base-pair to a second DNA or RNA molecule that contains a complementary sequence (the “target”). The stability of the resulting hybrid depends upon the extent of the base-pairing that occurs. The extent of base-pairing is affected by parameters such as the degree of complementarity between the probe and target molecules and the degree of stringency of the hybridization conditions. The degree of hybridization stringency is affected by parameters such as temperature, salt concentration, and the concentration of organic molecules such as formamide, and is determined by methods known to one skilled in the art. Probes or primers specific for NOD2 nucleic acids (for example, genes and/or mRNAs) have at least 80%-90% sequence complementarity, preferably at least 91%-95% sequence complementarity, more preferably at least 96%-99% sequence complementarity, and most preferably 100% sequence complementarity to the region of the NOD2 nucleic acid to which they hybridize. Probes, primers, and oligonucleotides may be detectably-labeled, either radioactively, or non-radioactively, by methods well-known to those skilled in the art. Probes, primers, and oligonucleotides are used for methods involving nucleic acid hybridization, such as: nucleic acid sequencing, reverse transcription and/or nucleic acid amplification by the polymerase chain reaction, single stranded conformational polymorphism (SSCP) analysis, restriction fragment polymorphism (RFLP) analysis, Southern hybridization, Northern hybridization, in situ hybridization, electrophoretic mobility shift assay (EMSA).

The terms “sample,” “patient sample,” “biological sample,” and the like, encompass a variety of sample types obtained from a patient, individual, or subject and can be used in a diagnostic or monitoring assay. The patient sample may be obtained from a healthy subject or a patient having symptoms associated with HCMV. Moreover, a sample obtained from a patient can be divided and only a portion may be used for diagnosis. Further, the sample, or a portion thereof, can be stored under conditions to maintain sample for later analysis. The definition specifically encompasses blood and other liquid samples of biological origin (including, but not limited to, peripheral blood, serum, plasma, cord blood, amniotic fluid, cerebrospinal fluid, urine, saliva, stool and synovial fluid), solid tissue samples such as a biopsy specimen or tissue cultures or cells derived therefrom and the progeny thereof. In certain embodiments, a sample comprises blood. In other embodiments, a sample comprises serum. In a specific embodiment, a sample comprises plasma. In another embodiment, a sample comprises urine. In yet another embodiment, a semen sample is used. In a further embodiment, a stool sample is used.

The definition of “sample” also includes samples that have been manipulated in any way after their procurement, such as by centrifugation, filtration, precipitation, dialysis, chromatography, treatment with reagents, washed, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry.

### II. NOD2 Promoter Mutations as Biomarkers

The present inventors have discovered that certain mutations in NOD1 and NOD2 are associated with HCMV susceptibility. In one embodiment, the NOD2 mutation is 3020insC. In other embodiments, the NOD2 mutation comprises one or more of R702W, G908R, L1007fs, and R334W. In further embodiments, mutations in one or more of vimentin, NOD1, OAS2, RIG-I, RIPK2, XIAP, Nemo (IKK gamma), IKK epsilon, IRF3, IRF5, and IRF7, can be used to identify HCMV susceptibility. In particular embodiments, the XIAP mutations comprise one or more of E99X, C203Y, G39C, K297T, and W323X. In another specific embodiment, the NOD1 mutation is E266K. The scope of the present invention includes all other known mutations of the foregoing genes, the sequences of which are known in the art and readily available.

Thus, in certain embodiments, the mutations can thus be used to identify individuals susceptible to or at risk of developing HCMV. Although the embodiments described herein might refer to mutations in NOD2, it is understood that such references include mutations in downstream or other NOD2-interacting proteins including, but not limited to, vimentin, NOD1, OAS2, RIG-I, RIPK2, XIAP, Nemo (IKK gamma), IKK epsilon, IRF3, IRF5, and IRF7.

In certain embodiments, DNA can be isolated from a biological sample taken from a subject. DNA can be extracted and purified from biological samples using any suitable technique. A number of techniques for DNA extraction and/or purification are known in the art, and several are commercially available (e.g., ChargeSwitch®, MELT™ total nucleic acid isolation system, MagMAX™ FFPE total nucleic acid isolation kit, MagMAX™ total nucleic acid isolation kit, QIAamp DNA kit, Omni-Pure™ genomic DNA purification system, WaterMaster™ DNA purification kit). Reagents such as DNAzoI® and TR1 Reagent® can also be used to extract and/or purify DNA. DNA can be further purified using Proteinase K and/or RNAse.

In further embodiments, primer/probes can be used to amplify a region of the NOD2 gene. More specifically, primers/probes are capable of amplifying a region of the NOD2 gene comprising 3020insC. Primer/probes can also be used to amplify regions of NOD1, OAS2, RIG-I, RIPK2, XIAP, Nemo (IKK gamma), IKK epsilon, IRF3, IRF5, and IRF7, including specifically regions that comprise the mutations described herein. Although particular embodiments may be described in the context of NOD2, it is understood that such embodiments can also refer to other genes described herein including, but not limited to, vimentin, NOD1, OAS2, RIG-I, RIPK2, XIAP, Nemo (IKK gamma), IKK epsilon, IRF3, IRF5, and IRF7.

In particular embodiments, a primer is contacted with isolated DNA from the subject under conditions such that the primer specifically hybridizes with the NOD2 gene. The primer and DNA thus form a primer:DNA complex. In certain embodiments, the hybridization conditions are such that the formation of the primer:DNA complex is the detection step itself, i.e., the complex forms only if the mutation (e.g., 3020insC) is present. In other embodiments, the primer:DNA complex is amplified using polymerase chain reaction, the presence (or not) of the mutation is detected. In certain embodiments, the mutations are detected by sequencing.

As described herein, in certain embodiments, the primers can used to support DNA amplification reactions. Typically the primers will be capable of being extended in a sequence specific manner. Extension of a primer in a sequence specific manner includes any methods wherein the sequence or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the polynucleotide sequences disclosed herein or region of the polynucleotide sequences disclosed herein or they hybridize with the complement of the polynucleotide sequences disclosed herein or complement of a region of the polynucleotide sequences disclosed herein.

The size of the primers or probes for interaction with the polynucleotide sequences disclosed herein in certain embodiments can be any size that supports the desired enzymatic manipulation of the primer, such as DNA amplification or the simple hybridization of the probe or primer. A typical primer or probe would be at least 6, 7, 8, 9, 10, 20, 30, 40, 50, 60, 70, 80, 90, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long or any length in-between.

The probes or primers of the present invention can be prepared by conventional techniques well-known to those skilled in the art. For example, the probes can be prepared using solid-phase synthesis using commercially available equipment. Modified oligonucleotides can also be readily prepared by similar methods. The probes can also be synthesized directly on a solid support according to methods standard in the art. This method of synthesizing polynucleotides is particularly useful when the polynucleotide probes are part of a nucleic acid array.

The present invention therefore also provides predictive, diagnostic, and prognostic kits comprising degenerate primers to amplify a target nucleic acid in the NOD2 gene (and/or downstream or other NOD2-interacting proteins) and instructions comprising amplification protocol and analysis of the results. The kit may alternatively also comprise buffers, enzymes, and containers for performing the amplification and analysis of the amplification products. The kit may also be a component of a screening, diagnostic or prognostic kit comprising other tools such as DNA microarrays. In some embodiments, the kit also provides one or more control templates, such as nucleic acids isolated from normal tissue sample, and/or a series of samples representing different variances in the NOD2 gene.

In one embodiment, the kit provides at least one primer capable of amplifying a different region of the NOD2 gene. The kit may comprise additional primers for the analysis of expression of several gene variances in a biological sample in one reaction or several parallel reactions. Primers in the kits may be labeled, for example fluorescently labeled, to facilitate detection of the amplification products and consequent analysis of the nucleic acid variances.

In one embodiment, more than one mutation/variance can be detected in one analysis. A combination kit will therefore comprise of primers capable of amplifying different segments of the NOD2 gene. A kit may also comprise primers capable of amplifying segments of another gene(s) including, but not limited to, vimentin, NOD1, OAS2, RIG-I, RIPK2, XIAP, Nemo (IKK gamma), IKK epsilon, IRF3, IRF5, and IRF7. The primers may be differentially labeled, for example, using different fluorescent labels, so as to differentiate between the variances. The primers contained within the kit may include primers selected from complementary sequences to the coding sequence of NOD2.

In certain embodiments, a patient can be diagnosed or identified by adding a biological sample (e.g., blood or blood serum) obtained from the patient to the kit and detecting the NOD2 mutations(s), for example, by a method which comprises the steps of: (i) collecting blood or blood serum from the patient; (ii) separating DNA from the patient's blood; (iii) adding the DNA from patient to a diagnostic kit; and, (iv) detecting (or not) the NOD2 mutation(s). In this exemplary method, primers are brought into contact with the patient's DNA. The formation of the primer:DNA complex can, for example, be PCR amplified and, in some embodiments, sequenced to detect (or not) the NOD2 mutation. In other kit and diagnostic embodiments, blood or blood serum need not be collected from the patient (i.e., it is already collected). Moreover, in other embodiments, the sample may comprise a tissue sample, urine or a clinical sample.

### III. NOD2 Pathway Modulators

In certain embodiments, the NOD2 Pathway modulator is selected from the group consisting of a small molecule, a polypeptide, a nucleic acid molecule, a peptidomimetic, or a combination thereof. In a specific embodiment, the agent can be a polypeptide. The polypeptide can, for example, comprise a domain of NOD2. The polypeptide can also comprise an antibody. In another embodiment, the agent can be a nucleic acid molecule. The nucleic acid molecule can, for example, be a NOD2 inhibitory nucleic acid molecule. The NOD2 inhibitory nucleic acid molecule can comprise a short interfering RNA (siRNA) molecule, a microRNA (miRNA) molecule, or an antisense molecule.

The term antibody is used herein in a broad sense and includes both polyclonal and monoclonal antibodies. The term can also refer to a human antibody and/or a humanized antibody. Examples of techniques for human monoclonal antibody production include those described by Cole et al. (Monoclonal Antibodies and Cancer Therapy, Alan R. Liss, p. 77 (1985)) and by Boerner et al. (J. Immunol. 147(1):86-95 (1991)). Human antibodies (and fragments thereof) can also be produced using phage display libraries (Hoogenboom et al., J. Mol. Biol. 227:381 (1991); Marks et al., J. Mol. Biol. 222:581 (1991)). The disclosed human antibodies can also be obtained from transgenic animals. For example, transgenic mutant mice that are capable of producing a full repertoire of human antibodies, in response to immunization, have been described (see, e.g., Jakobovits et al., Proc. Natl. Acad. Sci. USA 90:2551-5 (1993); Jakobovits et al., Nature 362:255-8 (1993); Bruggermann et al., Year in Immunol. 7:33 (1993)).

In other embodiments, a NOD2 pathway modulator is a small molecule. The term “small molecule organic compounds” refers to organic compounds generally having a molecular weight less than about 5000, 4000, 3000, 2000, 1000, 800, 600, 500, 250 or 100 Daltons, preferably less than about 500 Daltons. A small molecule organic compound may be prepared by synthetic organic techniques, such as by combinatorial chemistry techniques, or it may be a naturally-occurring small molecule organic compound.

Compound libraries may be screened for NOD2 pathway modulators. A compound library is a mixture or collection of one or more putative modulators generated or obtained in any manner. Any type of molecule that is capable of interacting, binding or has affinity for NOD2 may be present in the compound library. For example, compound libraries screened using this invention may contain naturally-occurring molecules, such as carbohydrates, monosaccharides, oligosaccharides, polysaccharides, amino acids, peptides, oligopeptides, polypeptides, proteins, receptors, nucleic acids, nucleosides, nucleotides, oligonucleotides, polynucleotides, including DNA and DNA fragments, RNA and RNA fragments and the like, lipids, retinoids, steroids, glycopeptides, glycoproteins, proteoglycans and the like; or analogs or derivatives of naturally-occurring molecules, such as peptidomimetics and the like; and non-naturally occurring molecules, such as “small molecule” organic compounds generated, for example, using combinatorial chemistry techniques; and mixtures thereof.

A library typically contains more than one putative modulator or member, i.e., a plurality of members or putative modulators. In certain embodiments, a compound library may comprise less than about 50,000, 25,000, 20,000, 15,000, 10000, 5000, 1000, 500 or 100 putative modulators, in particular from about 5 to about 100, 5 to about 200, 5 to about 300, 5 to about 400, 5 to about 500, 10 to about 100, 10 to about 200, 10 to about 300, 10 to about 400, 10 to about 500, 10 to about 1000, 20 to about 100, 20 to about 200, 20 to about 300, 20 to about 400, 20 to about 500, 20 to about 1000, 50 to about 100, 50 to about 200, 50 to about 300, 50 to about 400, 50 to about 500, 50 to about 1000, 100 to about 200, 100 to about 300, 100 to about 400, 100 to about 500, 100 to about 1000, 200 to about 300, 200 to about 400, 200 to about 500, 200 to about 1000, 300 to about 500, 300 to about 1000, 300 to 2000, 300 to 3000, 300 to 5000, 300 to 6000, 300 to 10,000, 500 to about 1000, 500 to about 2000, 500 to about 3000, 500 to about 5000, 500 to about 6000, or 500 to about 10,000 putative modulators. In particular embodiments, a compound library may comprise less than about 50,000, 25,000, 20,000, 15,000, 10,000, 5,000, 1000, or 500 putative modulators.

A compound library may be prepared or obtained by any means including, but not limited to, combinatorial chemistry techniques, fermentation methods, plant and cellular extraction procedures and the like. A library may be obtained from synthetic or from natural sources such as for example, microbial, plant, marine, viral and animal materials. Methods for making libraries are well-known in the art. See, for example, E. R. Felder, Chimia 1994, 48, 512-541; Gallop et al., J. Med. Chem. 1994, 37, 1233-1251; R. A. Houghten, Trends Genet. 1993, 9, 235-239; Houghten et al., Nature 1991, 354, 84-86; Lam et al., Nature 1991, 354, 82-84; Carell et al., Chem. Biol. 1995, 3, 171-183; Madden et al., Perspectives in Drug Discovery and Design 2, 269-282; Cwirla et al., Biochemistry 1990, 87, 6378-6382; Brenner et al., Proc. Natl. Acad. Sci. USA 1992, 89, 5381-5383; Gordon et al., J. Med. Chem. 1994, 37, 1385-1401; Lebl et al., Biopolymers 1995, 37 177-198; and references cited therein. Compound libraries may also be obtained from commercial sources including, for example, from Maybridge, ChemNavigator.com, Timtec Corporation, ChemBridge Corporation, A-Syntese-Biotech ApS, Akos-SC, G & J Research Chemicals Ltd., Life Chemicals, Interchim S.A., and Spectrum Info. Ltd.

### IV. Functional Assays

The functional characteristics of NOD2 pathway modulators can be tested in vitro and in vivo. NOD2 pathway modulators (e.g., small molecules) may be tested by their ability to inhibit HCMV replication. Modulators can also be tested for the ability to interfere with NOD2's (or upstream/downstream pathway member's) ability to bind its natural ligands and NOD2 pathway members, or to modulate certain biological processes.

NOD2 (or pathway members) binding to ligands can be detected using Biacore® by immobilizing ligands to a solid support and detecting soluble NOD2 binding thereto. Alternatively, NOD2 can be immobilized, and the ligand binding thereto can be detected. NOD2/ligand binding can also be analyzed by ELISA (e.g., by detecting NOD2 binding to immobilized ligands), or by fluorescence resonance energy transfer (FRET). To perform FRET, fluorophore-labeled NOD2 binding to ligands in solution can be detected (see, for example, U.S. Pat. No. 5,631,169).

NOD2-ligand binding can also be detected via “liquid binding” methods, i.e., measuring affinity in liquid setting, instead of in an immobilized environment. Such methods are offered by Roche. NOD2-ligand binding can also be detected by coimmunoprecipitation (Lagace et al., 2006 J. Clin. Inv. 116(11):2995-3005). To examine NOD2-ligand binding in this manner, HepG2 cells are cultured in sterol-depleted medium for 18 hours. Purified NOD2 is added to the medium in the presence of 0.1 mM chloroquine and the cells are incubated for one hour. Cells are lysed in mild detergent (1% digitonin w/vol). NOD2 or a ligand is immunoprecipitated from cell lysates, separated by SDS-PAGE, and immunoblotted to detect the presence of coimmunoprecipitated the ligand or NOD2, respectively (Lagace et al., 2006 J. Clin. Inv. 116(11):2995-3005). These assays may be conducted with a mutant form of NOD2 that binds to the ligand with a higher avidity (Lagace et al., 2006, supra).

NOD2 pathway modulators can be tested for the ability to increase or decrease ligand levels within the cells. For example, cells are cultured in sterol-depleted medium (DMEM supplemented with 100 U/ml penicillin, 100 μg/ml streptomycin sulfate, and 1 g/l glucose, 5% (vol/vol) newborn calf lipoprotein-deficient serum (NCLPDS), 10 μM sodium compactin, and 50 μM sodium mevalonate) for 18 hours to induce ligand expression. Purified NOD2 (about 5 μg/ml) is added to the medium. Ligand levels in cells harvested at 0, 0.5, 1, 2, and 4 hours after addition of NOD2 is determined (Lagace et al., 2006 J. Clin. Inv. 116(11):2995-3005). Ligand levels can be determined by flow cytometry, FRET, immunoblotting, or other means.

### V. Methods of Using NOD2 Pathway Modulators

The NOD2 pathway modulators described herein have in vitro and in vivo diagnostic and therapeutic utilities. For example, these molecules can be administered to cells in culture, e.g., in vitro or in vivo, or in a subject, e.g., in vivo, to treat, prevent or diagnose HCMV or other disease, disorder or condition that may be affected or mediated by NOD2. In a specific embodiment, the disease, disorder or condition is infection with human cytomegalovirus. NOD2 pathway modulators are particularly suitable for treating human patients suffering from HCMV.

### VI. Pharmaceutical Compositions and Administration

Accordingly, a pharmaceutical composition of the present invention may comprise an effective amount of a NOD2 pathway modulator. As used herein, the term “effective,” means adequate to accomplish a desired, expected, or intended result. More particularly, an “effective amount” or a “therapeutically effective amount” is used interchangeably and refers to an amount of a NOD2 pathway modulator, perhaps in further combination with yet another therapeutic agent, necessary to provide the desired “treatment” (defined herein) or therapeutic effect, e.g., an amount that is effective to prevent, alleviate, treat or ameliorate symptoms of a disease or prolong the survival of the subject being treated. In particular embodiments, the pharmaceutical compositions of the present invention are administered in a therapeutically effective amount to treat patients suffering from HCMV. As would be appreciated by one of ordinary skill in the art, the exact low dose amount required will vary from subject to subject, depending on age, general condition of the subject, the severity of the condition being treated, the particular compound and/or composition administered, and the like. An appropriate “therapeutically effective amount” in any individual case can be determined by one of ordinary skill in the art by reference to the pertinent texts and literature and/or by using routine experimentation.

The pharmaceutical compositions of the present invention are in biologically compatible form suitable for administration in vivo for subjects. The pharmaceutical compositions can further comprise a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which a NOD2 pathway modulator is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water may be a carrier when the pharmaceutical composition is administered orally. Saline and aqueous dextrose may be carriers when the pharmaceutical composition is administered intravenously. Saline solutions and aqueous dextrose and glycerol solutions may be employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried slim milk, glycerol, propylene, glycol, water, ethanol and the like. The pharmaceutical composition may also contain minor amounts of wetting or emulsifying agents, or pH buffering agents.

The pharmaceutical compositions of the present invention can take the form of solutions, suspensions, emulsions, tablets, pills, capsules, powders, sustained-release formulations and the like. The composition can be formulated as a suppository, with traditional binders and carriers such as triglycerides. Oral formulation may include standard carriers such as pharmaceutical grades of mannitol, lactose, starch, magnesium stearate, sodium saccharine, cellulose, magnesium carbonate, etc. In a specific embodiment, a pharmaceutical composition comprises an effective amount of a NOD2 pathway modulator together with a suitable amount of a pharmaceutically acceptable carrier so as to provide the form for proper administration to the patient. The formulation should suit the mode of administration.

The pharmaceutical compositions of the present invention may be administered by any particular route of administration including, but not limited to oral, parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intraosseous, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, iontophoretic means, or transdermal means. Most suitable routes are oral administration or injection. In certain embodiments, subcutaneous injection is preferred.

In general, the pharmaceutical compositions comprising a NOD2 pathway modulator may be used alone or in concert with other therapeutic agents at appropriate dosages defined by routine testing in order to obtain optimal efficacy while minimizing any potential toxicity. The dosage regimen utilizing a pharmaceutical composition of the present invention may be selected in accordance with a variety of factors including type, species, age, weight, sex, medical condition of the patient; the severity of the condition to be treated; the route of administration; the renal and hepatic function of the patient; and the particular pharmaceutical composition employed. A physician of ordinary skill can readily determine and prescribe the effective amount of the pharmaceutical composition (and potentially other agents including therapeutic agents) required to prevent, counter, or arrest the progress of the condition.

Optimal precision in achieving concentrations of the therapeutic regimen (e.g., pharmaceutical compositions comprising a NOD2 pathway modulator, optionally in combination with another therapeutic agent) within the range that yields maximum efficacy with minimal toxicity may require a regimen based on the kinetics of the pharmaceutical composition's availability to one or more target sites. Distribution, equilibrium, and elimination of a pharmaceutical composition may be considered when determining the optimal concentration for a treatment regimen. The dosages of a pharmaceutical composition disclosed herein may be adjusted when combined to achieve desired effects. On the other hand, dosages of the pharmaceutical compositions and various therapeutic agents may be independently optimized and combined to achieve a synergistic result wherein the pathology is reduced more than it would be if either was used alone.

In particular, toxicity and therapeutic efficacy of a pharmaceutical composition disclosed herein may be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effect is the therapeutic index and it may be expressed as the ratio LD₅₀/ED₅₀. Pharmaceutical compositions exhibiting large therapeutic indices are preferred except when cytotoxicity of the composition is the activity or therapeutic outcome that is desired. Although pharmaceutical compositions that exhibit toxic side effects may be used, a delivery system can target such compositions to the site of affected tissue in order to minimize potential damage to uninfected cells and, thereby, reduce side effects. Generally, the pharmaceutical compositions of the present invention may be administered in a manner that maximizes efficacy and minimizes toxicity.

Data obtained from cell culture assays and animal studies may be used in formulating a range of dosages for use in humans. The dosages of such compositions lie preferably within a range of circulating concentrations that include the ED₅₀ with little or no toxicity. The dosage may vary within this range depending upon the dosage form employed and the route of administration utilized. For any composition used in the methods of the invention, the therapeutically effective dose may be estimated initially from cell culture assays. A dose may be formulated in animal models to achieve a circulating plasma concentration range that includes the IC₅₀ (the concentration of the test composition that achieves a half-maximal inhibition of symptoms) as determined in cell culture. Such information may be used to accurately determine useful doses in humans. Levels in plasma may be measured, for example, by high performance liquid chromatography.

Moreover, the dosage administration of the compositions of the present invention may be optimized using a pharmacokinetic/pharmacodynamic modeling system. For example, one or more dosage regimens may be chosen and a pharmacokinetic/pharmacodynamic model may be used to determine the pharmacokinetic/pharmacodynamic profile of one or more dosage regimens. Next, one of the dosage regimens for administration may be selected which achieves the desired pharmacokinetic/pharmacodynamic response based on the particular pharmacokinetic/pharmacodynamic profile. See WO 00/67776, which is entirely expressly incorporated herein by reference.

More specifically, the pharmaceutical compositions may be administered in a single daily dose, or the total daily dosage may be administered in divided doses of two, three, or four times daily. In the case of oral administration, the daily dosage of the compositions may be varied over a wide range from about 0.1 ng to about 1,000 mg per patient, per day. The range may more particularly be from about 0.001 ng/kg to 10 mg/kg of body weight per day, about 0.1-100 μg, about 1.0-50 μg or about 1.0-20 mg per day for adults (at about 60 kg).

The daily dosage of the pharmaceutical compositions may be varied over a wide range from about 0.1 ng to about 1000 mg per adult human per day. For oral administration, the compositions may be provided in the form of tablets containing from about 0.1 ng to about 1000 mg of the composition or 0.1, 0.2, 0.5, 1.0, 2.0, 5.0, 10.0, 15.0, 100, 150, 200, 250, 300, 350, 400, 450, 500, 550, 600, 650, 700, 800, 900, or 1000 milligrams of the composition for the symptomatic adjustment of the dosage to the patient to be treated. An effective amount of the pharmaceutical composition is ordinarily supplied at a dosage level of from about 0.1 ng/kg to about 20 mg/kg of body weight per day. In one embodiment, the range is from about 0.2 ng/kg to about 10 mg/kg of body weight per day. In another embodiment, the range is from about 0.5 ng/kg to about 10 mg/kg of body weight per day. The pharmaceutical compositions may be administered on a regimen of about 1 to about 10 times per day.

In the case of injections, it is usually convenient to give by an intravenous route in an amount of about 0.0001 μg-30 mg, about 0.01 μg-20 mg or about 0.01-10 mg per day to adults (at about 60 kg). In the case of other animals, the dose calculated for 60 kg may be administered as well.

Doses of a pharmaceutical composition of the present invention can optionally include 0.0001 μg to 1,000 mg/kg/administration, or 0.001 μg to 100.0 mg/kg/administration, from 0.01 μg to 10 mg/kg/administration, from 0.1 μg to 10 mg/kg/administration, including, but not limited to, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53,54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100-500 mg/kg/administration or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration or any range, value or fraction thereof.

As a non-limiting example, treatment of subjects can be provided as a one-time or periodic dosage of a composition of the present invention 0.1 ng to 100 mg/kg such as 0.0001, 0.001, 0.01, 0.1 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Specifically, the pharmaceutical compositions of the present invention may be administered at least once a week over the course of several weeks. In one embodiment, the pharmaceutical compositions are administered at least once a week over several weeks to several months. In another embodiment, the pharmaceutical compositions are administered once a week over four to eight weeks. In yet another embodiment, the pharmaceutical compositions are administered once a week over four weeks.

More specifically, the pharmaceutical compositions may be administered at least once a day for about 2 days, at least once a day for about 3 days, at least once a day for about 4 days, at least once a day for about 5 days, at least once a day for about 6 days, at least once a day for about 7 days, at least once a day for about 8 days, at least once a day for about 9 days, at least once a day for about 10 days, at least once a day for about 11 days, at least once a day for about 12 days, at least once a day for about 13 days, at least once a day for about 14 days, at least once a day for about 15 days, at least once a day for about 16 days, at least once a day for about 17 days, at least once a day for about 18 days, at least once a day for about 19 days, at least once a day for about 20 days, at least once a day for about 21 days, at least once a day for about 22 days, at least once a day for about 23 days, at least once a day for about 24 days, at least once a day for about 25 days, at least once a day for about 26 days, at least once a day for about 27 days, at least once a day for about 28 days, at least once a day for about 29 days, at least once a day for about 30 days, or at least once a day for about 31 days.

Alternatively, the pharmaceutical compositions may be administered about once every day, about once every 2 days, about once every 3 days, about once every 4 days, about once every 5 days, about once every 6 days, about once every 7 days, about once every 8 days, about once every 9 days, about once every 10 days, about once every 11 days, about once every 12 days, about once every 13 days, about once every 14 days, about once every 15 days, about once every 16 days, about once every 17 days, about once every 18 days, about once every 19 days, about once every 20 days, about once every 21 days, about once every 22 days, about once every 23 days, about once every 24 days, about once every 25 days, about once every 26 days, about once every 27 days, about once every 28 days, about once every 29 days, about once every 30 days, or about once every 31 days.

The pharmaceutical compositions of the present invention may alternatively be administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 weeks, about once every 20 weeks.

Alternatively, the pharmaceutical compositions of the present invention may be administered about once every month, about once every 2 months, about once every 3 months, about once every 4 months, about once every 5 months, about once every 6 months, about once every 7 months, about once every 8 months, about once every 9 months, about once every 10 months, about once every 11 months, or about once every 12 months.

Alternatively, the pharmaceutical compositions may be administered at least once a week for about 2 weeks, at least once a week for about 3 weeks, at least once a week for about 4 weeks, at least once a week for about 5 weeks, at least once a week for about 6 weeks, at least once a week for about 7 weeks, at least once a week for about 8 weeks, at least once a week for about 9 weeks, at least once a week for about 10 weeks, at least once a week for about 11 weeks, at least once a week for about 12 weeks, at least once a week for about 13 weeks, at least once a week for about 14 weeks, at least once a week for about 15 weeks, at least once a week for about 16 weeks, at least once a week for about 17 weeks, at least once a week for about 18 weeks, at least once a week for about 19 weeks, or at least once a week for about 20 weeks.

Alternatively the pharmaceutical compositions may be administered at least once a week for about 1 month, at least once a week for about 2 months, at least once a week for about 3 months, at least once a week for about 4 months, at least once a week for about 5 months, at least once a week for about 6 months, at least once a week for about 7 months, at least once a week for about 8 months, at least once a week for about 9 months, at least once a week for about 10 months, at least once a week for about 11 months, or at least once a week for about 12 months.

The pharmaceutical compositions may further be combined with one or more additional therapeutic agents. In particular embodiments, the second therapeutic agent can be an antiviral. A combination therapy regimen may be additive, or it may produce synergistic results.

The compositions can be administered simultaneously or sequentially by the same or different routes of administration. The determination of the identity and amount of the pharmaceutical compositions for use in the methods of the present invention can be readily made by ordinarily skilled medical practitioners using standard techniques known in the art. In specific embodiments, a NOD2 pathway modulator of the present invention can be administered in combination with an effective amount of another therapeutic agent, depending on the disease or condition being treated.

In various embodiments, the NOD2 pathway modulator of the present invention in combination with an another therapeutic agent may be administered at about the same time, less than 1 minute apart, less than 2 minutes apart, less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In particular embodiments, two or more therapies are administered within the same patent visit.

In certain embodiments, the NOD2 pathway modulator of the present invention in combination with another therapeutic agent are cyclically administered. Cycling therapy involves the administration of a first therapy (e.g., the NOD2 pathway modulator) for a period of time, followed by the administration of a second therapy (e.g., another therapeutic agent) for a period of time, optionally, followed by the administration of perhaps a third therapy for a period of time and so forth, and repeating this sequential administration, e.g., the cycle, in order to reduce the development of resistance to one of the therapies, to avoid or reduce the side effects of one of the therapies, and/or to improve the efficacy of the therapies. In certain embodiments, the administration of the combination therapy of the present invention may be repeated and the administrations may be separated by at least 1 day, 2 days, 3 days, 5 days, 10 days, 15 days, 30 days, 45 days, 2 months, 75 days, 3 months, or at least 6 months.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

Ethics Statement: Clinical isolates of HCMV and herpesvirus 2 (HSV2) were obtained from the microbiology laboratory at Johns Hopkins Hospital with no identifiers that could be linked to a patient. The Johns Hopkins School of Medicine Office of Human Subject Research Institutional Review Board (IRB-X) determined that the research qualified for an exemption.

Cell culture and Viruses: Human Foreskin Fibroblasts (HFFs) passage 12-16 (ATCC, CRL-2088) and U373 glioma cells (provided by Dr. Gary Hayward, Johns Hopkins Medical Institutions [31]) were grown in Dulbecco's Modified Eagle Medium (DMEM) containing 10% fetal bovine serum (FBS) (Gibco, Carlsbad, Calif.) in a 5% CO₂ incubator at 37° C. and used for infection with several HCMV strains. One day prior to infection, 8×10⁴ HFFs or U373 cells were seeded on each well of 12-well tissue culture plates. Infection was carried out at multiplicity of infection of 1 PFU/cell (MOI=1) unless otherwise specified. The pp28-luciferase HCMV Towne strain which expresses luciferase under the control of pp28 late promoter has been described and was shown to correlate well with the classic plaque assay [32]. HCMV strain TB40 with the UL32 gene fused to GFP was obtained from ATCC (VR-1578). Clinical isolates of HCMV and HSV2 were obtained from the microbiology laboratory at Johns Hopkins Hospital with no identifiers that could be linked to a patient. A luciferase-tagged HSV1 (KOS/Dlux/oriS) was provided by Dr. David Leib, Dartmouth Medical School.

Ultraviolet (UV) inactivation of HCMV: HCMV was UV inactivated by spreading a thin layer of stock suspension in an uncovered six-well tissue culture plate and exposing to a total dose of 720 mJ/cm² in a UV crosslinker (Spectrolinker XL-1000, Spectronics, Westbury, N.Y.) [33]. Luciferase activity was measured in cell lysates of UV inactivated HCMV-infected HFFs 72 hours post infection (hpi), to quantify the level of inactivation. Luciferase units measured from the UV-inactivated HCMV were similar to those measured in the negative control wells, confirming near-complete virus inactivation.

Chemicals and proteins: MDP was obtained from Sigma Chemicals, (St. Louis, Mo.) and dissolved in PBS to prepare a stock of 10 mg/ml. Recombinant HCMV gB was purchased from DevaTal Inc. (Hamilton, N.J.).

Plasmids, transfections and virus replication assays: U373 cells were transfected with the following plasmids using Lipofectamine 2000 (Invitrogen, Carlsbad, Calif.): Human RIPK2 (pcDNA4/HisMax-hRIPK2), human NOD2 (pcDNA4/Hismax-hNOD2) and control pcDNA4/HisMax, (kindly provided by Dr. Michael Davey, Oregon Health and Science University). A pcDNA4-EGFP plasmid was used as additional control. NOD2 3020insC cDNA was PCR amplified from 3020insC-pEF6V5 plasmid (provided by Dr. Jurgen Harder, University Hospital Schleswig-Holstein, Campus Kiel, Germany) and subcloned into BamH1 and Xho1 site of pcDNA4/HisMax plasmid to generate pcDNA4/HisMax-hNOD2 3020insC construct. For amplification of NOD2 3020insC cDNA the following primers were used: 5′-GGATCCATGGGGGAAGAGGGTGGTTC-3′ (Forward) (SEQ ID NO:1) and 5′-CTCGAGTCAAAGCAAGAGTCTGGTGTCCC-3′ (Reverse) (SEQ ID NO:2). Restriction enzyme sites are underlined. The PCR protocol included preheating at 98° C. for 30 s, followed by 35 cycles of 98° C. (10 s), 60° C. (30 s) and 72° C. (2 min) PCR products were purified, cleaved by BamH1 and Xho1 and ligated into BamH1 and Xho1 cleaved pcDNA4/HisMax plasmid. The sequence of developed constructs was confirmed by DNA sequencing at the synthesis and sequencing facility, Johns Hopkins University.

Transient transfections of U373 cells were performed in 12-well plates with 1 μg/well of each plasmid using Lipofectamine 2000. After overnight transfection media were changed and cells were allowed to grow for another 24 hours before infection with pp28-luciferase HCMV. Luciferase activity (a measure of late HCMV gene expression) was determined at 96 hpi. We reported that the pp28-luciferase assay correlates well with plaque reduction [32]. DNA replication in HCMV-infected U373 cells and virus DNA yield in supernatants from U373 were measured using real-time PCR as previously below [34]. Virus DNA yield in supernatants from HCMV-infected HFFs were measured using real-time PCR. Virus DNA yield was also measured in supernatants of fresh HFFs after second cycle infection using supernatants from NOD2 knockdown and NOD2 control HFFs.

Generation of recombinant lentiviral vectors and establishment of stable cell lines: Stable cell lines overexpressing human NOD2 and control plasmids were generated using a doxycycline-inducible TRIPZ lentiviral vector (Open Biosystems, Huntsville, Ala.). The pMACS Kk hNOD2 HA(C) vector encoding full length human NOD2 (provided by Dr. Atsushi Kitani, NIAID/NIH) was used to prepare NOD2-TRIPZ expression constructs. NOD2 cDNA was subcloned into AgeI and MluI sites of pTRIPZ vector to create pTRIPZ-NOD2 construct. NOD2 cDNA was amplified by PCR using the following primers: 5′-GGGATCCACCGGTCCACCATGGGGGAAGAG-3′ (Forward) (SEQ ID NO:3) and 5′-GGGATCCACGCGTTCATTAAGCGTAGTCTGGGACGTC-3′ (Reverse) (SEQ ID NO:4). Following preheating at 98° C. for 30 s, the conditions used for PCR were, 98° C. (10 s), 55° C. (30 s), 72° C. (2 min), for 35 cycles. PCR products were purified, digested with AgeI and MluI, and ligated to pTRIPZ vector to generate hNOD2-TRIPZ construct. The sequence of the new constructs was confirmed by DNA sequencing. hNOD2-TRIPZ and TRIPZ control empty vector were packaged using lentivirus, as described below for the knockdown procedure. To generate stable HFF cell lines expressing hNOD2 or control plasmid, the lentivirus particles were transduced into HFFs. 0.5×10⁶ cells were plated onto T-25 flask, and 40 μl of concentrated virus and Polybrene at final concentration of 8 μg/ml were added to the cells, and incubated for 4 h. 48 h following transduction puromycin (2 μg/ml) containing media was added to culture flasks to select for stably transduced cells. NOD2 over-expressing and control cells were counted and an equal number of cells was seeded into each well prior to infection. An MTT assay (Sigma-Aldrich, St. Louis, Mo.) was performed to rule out cell toxicity following 48 h doxycyline induction. After the addition of 20 μl/well of MTT (3-(4,5-Dimethyl-2-thiazolyl)-2,5-diphenyl-2H-tetrazolim bromide) (5 mg/ml in PBS), and shaking at 150 rpm for 5 minutes the plates were incubated at 37° C. for 3 hours. Conversion of yellow solution to dark blue formazan by mitochondrial dehydrogenases of living cells was quantified by measuring absorbance at 560 nM.

Lentivirus-mediated Knockdown (KD) of NOD2: Human GIPZ lentiviral shRNAmir constructs (Open Biosystems) were used for NOD2 KD in U373 and HFFs. Four clones (clone id: V2LHS_225438, V3LHS_11832, V3LHS_365839, and V3LHS_365841) targeting different regions of NOD2 mRNA were tested for KD efficiency, and the clone with the best KD efficiency was selected to generate stable cell lines. GIPZ non-targeting control plasmid was used to rule out non-specific effects of shRNAmir constructs. Individual shRNAmir constructs were packaged using lentivirus as described [35]. Briefly, 21 μg of gag/pol, 7 μg of vesicular stomatitis virus glycoprotein, and 7 μg of shRNAmir plasmids were transfected into HEK293 cells using calcium phosphate method. After 48 h the packaged lentivirus particles were concentrated from the medium. The supernatant was filtered and centrifuged at 1750 g for 30 min at 4° C. in Amicon Ultra (Ultracel 100 k, Millipore). After centrifugation, 2 ml of cold PBS was added and the tubes were centrifuged again for 20 min at 4° C. The concentrated virus was stored at −80° C. until used. Lentivirus particles containing shRNAmir were transduced into HFFs or U373 cells. 0.5×10⁶ cells were plated onto T-25 flask and 40 μl of concentrated virus and polybrene (final concentration, 8 μg/ml) were added to the cells, and incubated for 4 h. Following transduction puromycin (2 μg/ml) was added to select for stably transduced cells. Control HFFs and NOD2 KD HFFs were counted and equal number of cells was plated into each well prior to infection.

RNA isolation and real time quantitative reverse transcriptase (qRT) PCR: Total RNA was isolated from cultured cells using RNeasy Mini kit (Qiagen, Georgetown, Md.) according to manufacturer's instructions. RevertAid first strand cDNA synthesis kit (Fermentas life sciences, Cromwell Park, Md.) was used to synthesize first strand cDNA from total RNA using oligo-dT primers. Negative reverse-transcriptase (−RT) reactions were included to ensure the specificity of qRT-PCR reactions. Synthesis of first strand cDNA from mRNA template was carried out at 42° C. for 1 h. Quantitative RT-PCR (qRT-PCR) was performed using specific primers and SYBER green (Fermentas life science) with two-step cycling protocol (95° C. for 15 s, 60° C. for 1 min). Reactions were performed in triplicates and GAPDH was used as internal control. mRNA levels in HCMV-infected cells were normalized to the mRNA produced in non-infected HFFs in addition to the internal normalization of each sample to GAPDH. The primers and gene targets appear in Table 1.

TABLE 1
Primers used for qualitative
and quantitative real-time PCR

| Gene | Forward | Reverse |
|---|---|---|
| NOD1 | 5′-CCTAGACAACAA | 5′-TTTACCCCACCG |
| | CAATCTCAACGACT | TCAGTGATC-3′ |
| | A-3′ | (SEQ ID NO: 6) |
| | (SEQ ID NO: 5) | |
| NOD2 | 5′-GCCACGGTGAAA | 5′-GGAAGCGAGACT |
| | GCGAAT-3′ | GAGCAGACA-3′ |
| | (SEQ ID NO: 7) | (SEQ ID NO: 8) |
| IL8 | 5′-TGCAGCTCTGT | 5′-CAGTGTGGTCC |
| | GTGAAGGTGCAG | ACTCTCAATCACT |
| | T-3′ | C-3′ |
| | (SEQ ID NO: 9) | (SEQ ID NO: 10) |
| IFNB | 5′-GATTCATCTAG | 5′-CTTCAGGTAATG |
| | CACTGGCTGG-3′ | CAGAATCC-3′ |
| | (SEQ ID NO: 11) | (SEQ ID NO: 12) |
| ISG15 | 5′-GCT CCA TGT | 5′-CTC GAA GGT |
| | CGG TGT CAG | CAG CCA GAA |
| | AG-3 | CAG-3′ |
| | (SEQ ID NO: 13) | (SEQ ID NO: 14) |
| GAPDH | 5′-TTGGTATCGTGG | 5′-ACAGTCTTCTGG |
| | AAGGACTC-3′ | GTGGCAGT-3′ |
| | (SEQ ID NO: 15) | (SEQ ID NO: 16) |
| Viperin | 5′-CAA GAG GAG | 5′-CAG GAG ATA |
| | AAA GCA GCA | GCG AGA ATG |
| | GC-3′ | TCC-3′ |
| | (SEQ ID NO: 17) | (SEQ ID NO: 18) |

Real-time PCR: HCMV DNA replication in cells and virus DNA yield in supernatants were quantified using a real-time PCR of the highly conserved US17 as previously described [32, 36].

SDS-polyacrylamide gel electrophoresis and immunoblot analysis: Cell lysates containing equivalent amount of proteins were mixed with an equal volume of sample buffer (125 mM Tris-HCL, pH 6.8, 4% SDS, 20% glycerol and 5% β-mercaptoethanol) and boiled at 100° C. for 10 min. Denatured proteins were resolved in Tris-glycine polyacrylamide gels (10-12%) and transferred to polyvinylidine difluoride (PVDF) membranes (Bio-Rad Laboratories, Hercules, Calif.) by electroblotting. Membranes were incubated in blocking solution [5% non-fat dry milk and 0.1% Tween-20 in PBS (PBST)] for 1 hr, washed with PBST, and incubated with antibody at 4° C. overnight. Membranes were washed with PBST and incubated with horseradish peroxidase-conjugated secondary antibodies in PBST for 1 hr at room temperature. Following washing with PBST, protein bands were visualized by chemiluminescence using SuperSignal West Dura and Pico reagents (Pierce Chemical, Rockford, Ill.). Antibodies for HCMV included: mouse anti-human CMV 1E1 & 1E2 (MAB810) (Millipore, Billerica, Mass., 1:2,000), mouse anti-human CMV UL83 (pp65) (Vector Laboratories Inc., Burlingame, Calif., 1:2,000), mouse anti-human CMV UL44 (10E8), mouse anti-human β-actin (Sigma, 1:5,000). For detecting NOD2, mouse anti-human NOD2 (2D9) antibody (Novus Biologicals, Littleton, Colo., 1:2000) and rabbit anti human-NOD2 (H-300) antibody (Santa Cruz Biotechnology Inc, Santa Cruz, Calif., 1:2000) were used. Mouse anti-NF-κB (p65, Sc-8008), rabbit anti-IRF3 antibody (FL-425, Sc-9082) (Santa Cruz biotechnology Santa Cruz, Calif., 1:2,000), and rabbit anti-Histone H3 (D1H2, #4499) antibody (Cell Signaling Technology, 1:2000) were used for detection of these proteins in cytoplasmic and nuclear extracts.

Preparation of cytosolic and nuclear extracts: Cytoplasmic and nuclear fractions were isolated from HFF-shNOD2 and HFF-control cells as previously reported with minor modifications [37]. Extracts were prepared from HCMV-infected or mock-infected cells at 24 hpi. Briefly, cells were washed twice with ice-cold phosphate-buffered saline (PBS) and resuspended on ice for 15 min in buffer A containing 10 mM HEPES (pH 7.9), 10 mM KCl, 0.1 mM EDTA, 1 mM dithiothreitol (DTT), protease and phosphatase inhibitors. Cells were then lysed by adding 0.1% NP40 and cytosolic supernatants were obtained by centrifugation at 10,000 rpm for 30 sec. Crude nuclei were washed twice with buffer A to prevent cytosolic contamination, and the nuclear proteins were extracted by resuspending cell pellets with buffer C containing 20 mM HEPES (pH 7.9), 400 mM NaCl, 1 mM EDTA, 1 mM DTT, protease and phosphatase inhibitors. The mixture was incubated for 15 min with vigorous shaking on rocker at 4° C. and then centrifuged at 14,000 rpm at 4° C. for 10 min to obtain the nuclear proteins. Protein concentration was determined using BCA protein assay reagent kit (Pierce Chemical, Rockford, Ill.).

ELISA: Human interferon β (IFN-β) specific ELISA kit (PBL Interferon Source) was used to measure levels of secreted IFN-β from HFF-control and HFF-NOD2 overexpressing cells according to manufacturer's instructions.

Yield Assay: Human lung fibroblast cells, MRC5, (Diagnostic Hybrids, Athens, Ohio, 51-0600) were used to perform a virus yield assay. Cells were seeded into 12 well plates (2×10⁵ cells/well) and infected using cell free supernatants collected 3 days post infection (dpi) from HCMV-infected HFF-GIPZ (control) or HFF-shNOD2 cells (second cycle infection). After 90 minute adsorption, media were aspirated, and DMEM containing 0.5% carboxymethyl-cellulose and 4% fetal bovine serum (FBS) were added into duplicate cells. After incubation at 37° C. for 8 days the overlay was removed and plaques were counted after crystal violet staining.

Statistical Analysis: Data were expressed as mean±SD of three or more independent experiments. The data were analyzed by one-way ANOVA comparisons between different groups with significance value set at P<0.05.

### Results

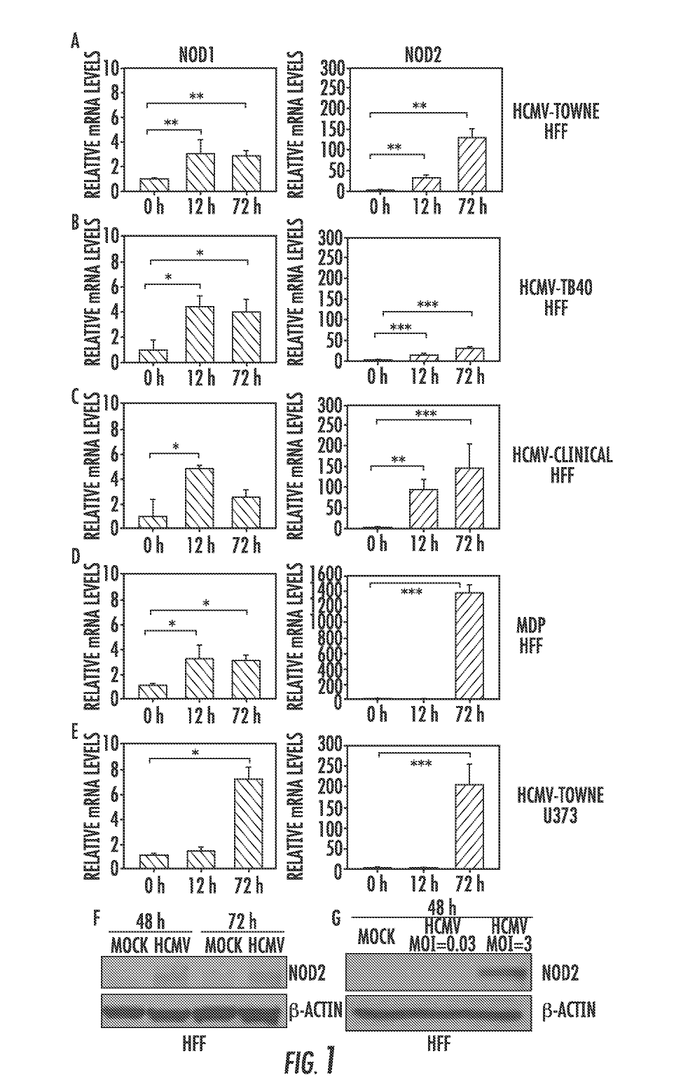

HCMV infection results in significant induction of NOD2 expression: mRNA levels of NOD1 and NOD2 were measured by qRT-PCR in HCMV-infected HFFs. The following HCMV strains were used for infection of HFFs at MOI of 1 PFU/cell: the laboratory-adapted strain, Towne, the endotheliotropic TB40-GFP strain (ATCC VR-1578), and a clinical isolate of HCMV. Infection with all three viruses (Towne, TB40 and a clinical isolate) resulted in robust induction of NOD2 transcripts at 12 and 72 hours post infection (hpi), while in non-infected HFFs NOD2 mRNA was undetectable (FIGS. 1A, B and C). Treatment of non-infected HFFs with MDP, a positive control for NOD2 induction [38], resulted in two fold induction of NOD2 expression at 12 h, and a potent NOD2 induction (1400-fold) at 72 h (FIG. 1D). Similarly, infection of U373 glioma cells with Towne HCMV resulted in significant upregulation of NOD2 at 72 hpi (FIG. 1E). In contrast to NOD2, NOD1 was already expressed in non-infected HFFs, and there was a modest increase (˜3-7 fold) in NOD1 transcripts at 12 and 72 hpi (FIG. 1A-E), depending on the HCMV strain and cell type used. NOD2 protein was upregulated in HCMV-infected HFFs at 48 and 72 hpi (FIG. 1F) and its expression was MOI dependent (FIG. 1G).

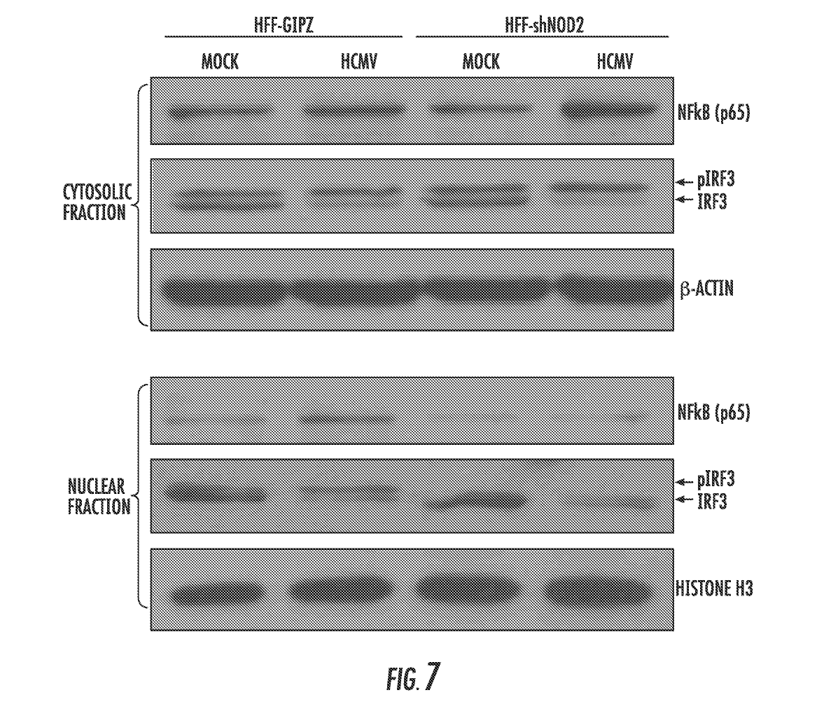

Infection with Herpesvirus (HSV1 and HSV2) does not induce NOD2: To determine whether NOD2 could be induced by other herpes viruses, HFFs were infected with herpesvirus 1KOS/Dlux/oriS and a clinical isolate of HSV-2 at MOI of 1 and 0.1, respectively. NOD1 and NOD2 transcripts were quantified at 2, 4, 6, 8 and 24 hpi in HSV-1 infected HFFs using qRT-PCR. There was no significant change in NOD1 expression between non-infected and HSV1-infected HFFs (FIG. 8A). NOD2 was again undetectable in non-infected HFFs, but was not induced after infection with HSV1. Similarly, there was no significant increase in NOD1 expression in HSV2-infected HFFs at 4 and 24 hpi as compared to non-infected HFFs (FIG. 8B) and NOD2 was undetectable in both non-infected and HSV2-infected HFFs.

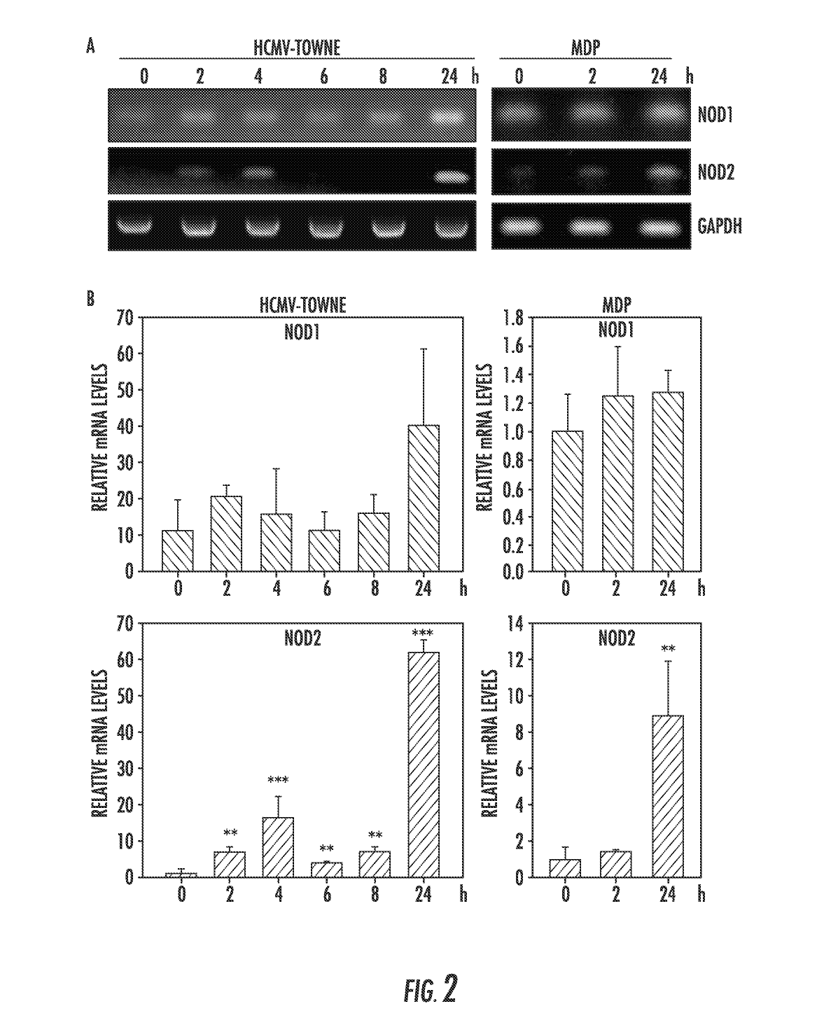

NOD2 is induced early after HCMV infection: To understand the kinetics of NOD2 induction early after HCMV infection a time-course experiment was performed in HFFs. NOD1 and NOD2 transcripts were quantified starting from the time of HCMV infection and then at 2, 4, 6, 8, and 24 hpi (FIGS. 2A, B). MDP was used as positive control for NOD2 induction. Upregulation of NOD2 mRNA was observed as early as 2 hpi and increased significantly at 24 hpi. NOD2 transcripts remained elevated at 72 hpi (FIG. 1 A-C). A modest increase in NOD1 transcripts (up to 5-fold) was observed at 24 hpi.

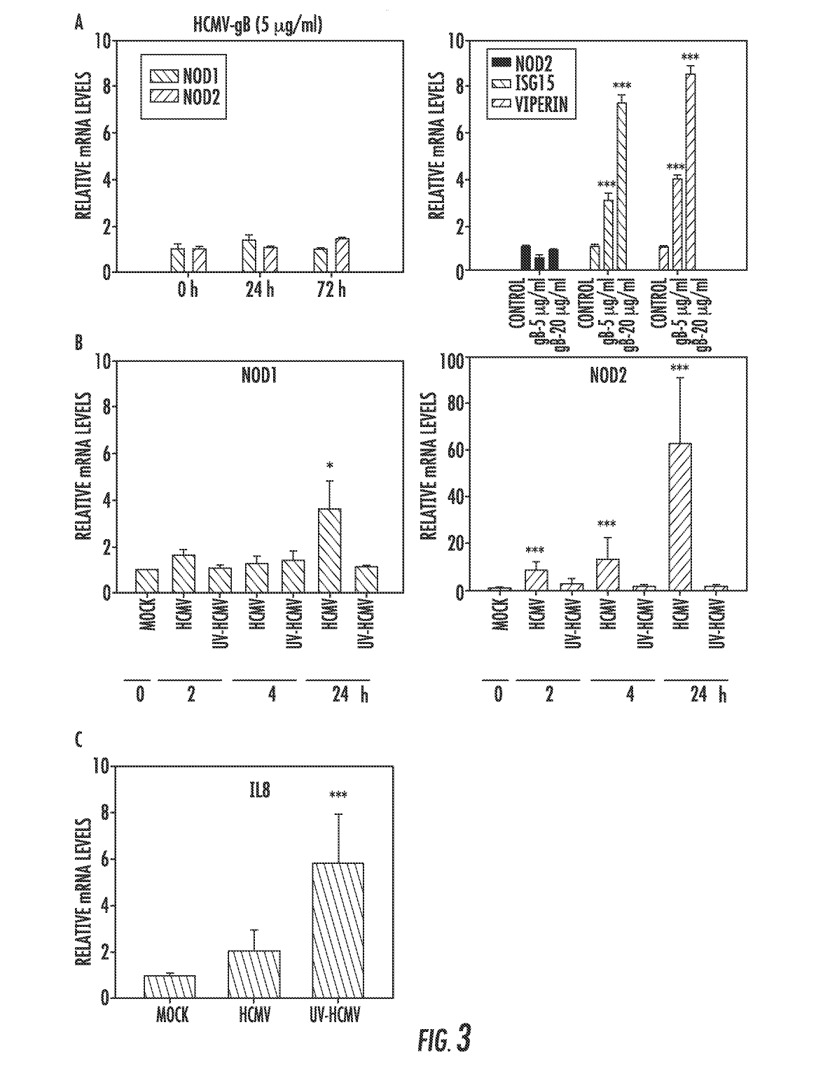

Glycoprotein B alone cannot induce NOD2 expression: Since HCMV-encoded gB was reported to bind to and activate TLR2 we tested whether it was required for NOD2 induction [10]. HFFs were treated with purified HCMV gB protein (5 μg/ml or 20 μg/ml, DevaTal, Hamilton, N.J.) and levels of NOD1 and NOD2 were measured at 24 and 72 hpi using qRT-PCR. Levels of NOD1 were unchanged in untreated vs. gB-treated HFFs. Treatment with gB alone did not induce NOD2 transcripts (FIG. 3A), but interferon inducible gene 15 (ISG15) and viperin were induced by gB.

UV-inactivated virus is unable to induce NOD2: To determine whether an intact HCMV genome was essential for NOD2 induction a UV-inactivated virus which could bind to the cells but could not replicate was used. The UV-inactivated virus did not induce NOD2 or NOD1 expression, suggesting an intact HCMV genome was required for induction of NOD2 (FIG. 3B). IL8 was induced (6-8 fold) using the UV-inactivated HCMV at 4 hpi, more efficiently than its induction by HCMV, in agreement with previous reports that suggested the induction of IL8 mRNA could be prevented by newly synthesized viral gene products [39].

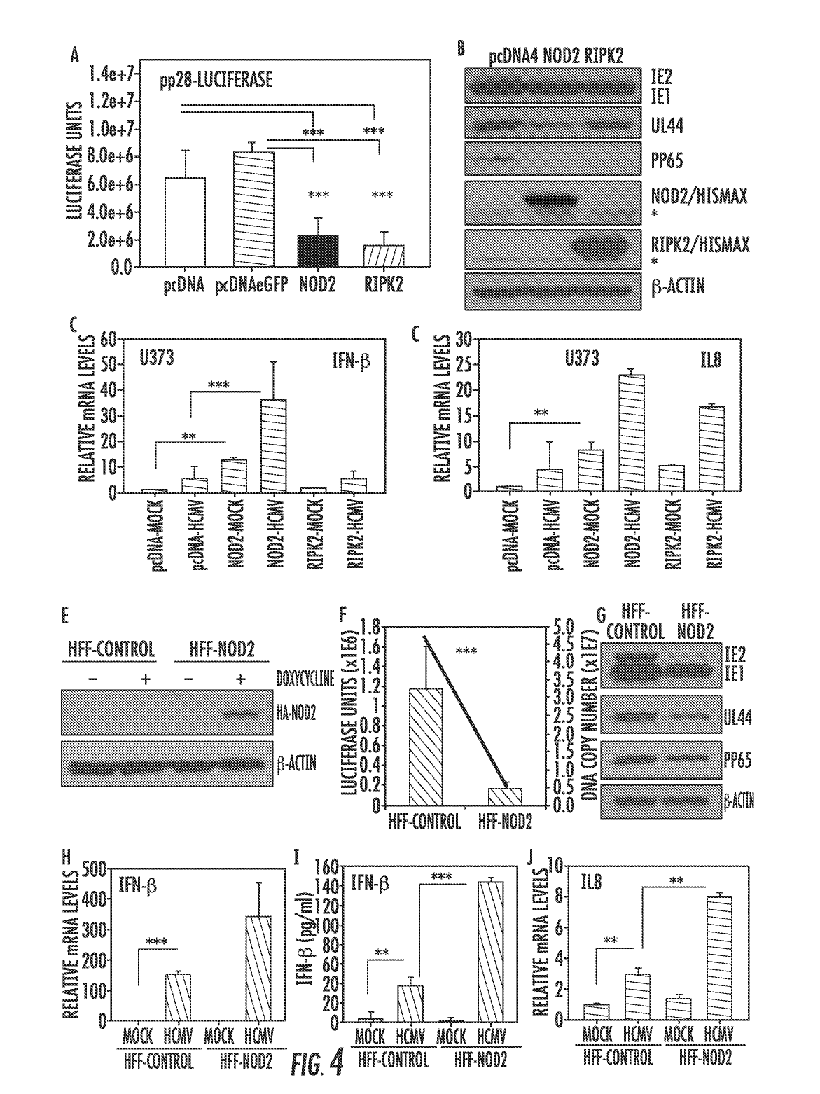

Overexpression of NOD2 results in decreased HCMV replication and enhanced antiviral and pro-inflammatory cytokine responses: HCMV induces multiple cellular genes to achieve efficient replication. We hypothesized that if NOD2 had a role in HCMV recognition and was not simply an induced gene amongst many other cellular genes, then its overexpression or KD would result is restricted or enhanced virus replication, respectively, along with changes in antiviral and pro-inflammatory cytokine responses. U373 glioma cells were initially used for transfections because the efficiency of transient transfection followed by HCMV infection in these cells was found to be high. Cells were transfected with human pcDNA4/HisMax-NOD2 or the pcDNA4/HisMax-RIPK2 (a critical kinase downstream of NOD2) plasmids and control plasmids pcDNA4/HisMax and pcDNA4-EGFP. 24 h following transfection cells were infected with HCMV at MOI=1 and luciferase activity was measured at 72 hpi. NOD2 overexpression resulted in ˜70% reduction of pp28 gene expression (FIG. 4A), consistent with significant inhibition of HCMV replication. In NOD2-transfected HCMV-infected cells the expression of HCMV immediate early 2 (IE2) and the early protein UL44 were significantly reduced as compared to control pcDNA4-transfected HCMV-infected cells. The expression of the late HCMV protein pp65 was completely undetectable in NOD2 overexpressing cells (FIG. 4B). Overexpression of RIPK2 resulted in significant inhibition of pp28-luciferase activity and pp65 expression, suggesting the effect of NOD2 in HCMV-infected cells may involve at least in part RIPK2 activity. Overexpression of NOD2 and RIPK2 was confirmed by western blot using anti-NOD2 and anti-RIPK2 antibodies, respectively (FIG. 4B). Levels of IFN-β and the inflammatory cytokine IL8 were measured by qRT-PCR in NOD2-, RIPK2- and control pcDNA4 plasmid-transfected cells. There was approximately ten- and three-fold increase in expression of IFN-β in NOD2 and RIPK2-overexpressing-HCMV infected cells as compared to HCMV infected cells transfected with pcDNA4 plasmid (FIG. 4C) at 72 hpi. Levels of IL8 mRNA were upregulated by approximately five- and three-fold in NOD2 and RIPK2-overexpressing-HCMV infected cells as compared to HCMV-infected cells transfected with pcDNA4 control plasmid (FIG. 4D) at 72 hpi. These results suggest that the effects of NOD2 on HCMV may involve both IFN-β and IL8.

To further understand the role of NOD2 in regulating HCMV replication, an inducible lentiviral pTRIPZ-based vector overexpressing HA-tagged NOD2 was generated in HFFs (HFF-NOD2). HFFs overexpressing control pTRIPZ empty vector (HFF-control) were also generated. NOD2 expression was confirmed by western blot analysis, performed after 48 h induction with doxycycline, and using anti-HA antibody (FIG. 4E). β-Actin was used as a loading control. HCMV replication was next measured in HFF-NOD2 and HFF-control cells. Doxycycline (2 μg/ml) induction was performed 24 h before infection with HCMV pp28-luciferase at an MOI of 0.5. Cells were counted and equal number of cells from each condition was seeded into wells prior to infection. To rule out potential toxicity secondary to induction, a MTT assay was performed 48 h after doxycycline induction and revealed no effect on cell viability. Cell-free supernatants were collected from infected HFFs-NOD2 and infected HFFs-control cells 4 days post infection (dpi) and were used for a second cycle infection of fresh HFFs at equivalent volumes. HCMV replication was then measured in the newly-infected HFFs using pp28-luciferase, virus DNA yield in supernatants of newly-infected HFFs and by western blots for HCMV proteins IE1, UL44 and pp65 (FIGS. 4F, G). At least 80% decrease was observed in luciferase expression and virus DNA yield in the second cycle of newly-infected HFFs using supernatants from HFF-NOD2 as compared to HCMV replication in newly-infected HFFs using supernatants from HFF-control cells, suggesting NOD2 induced a cellular immune state that was refractory to HCMV replication. Similarly, there was a significant decrease in the expression of IE1, UL44 and pp65 in HFFs infected with supernatants from HFF-NOD2 transduced cells as compared to HFF-control cells. The observed changes in HCMV replication were associated with changes in the transcripts of IFN-β and IL8, measured at 96 hpi in HFF-NOD2 and HFF-control cells (first cycle). There was approximately two and three-fold increase in the expression of IFN-β and IL8 mRNA, respectively, in HFFs-NOD2 as compared to HFFs-control (FIGS. 4H, J). Levels of IFN-β protein secreted into the media from non-infected and HCMV-infected-HFFs-control and HFFs-NOD2 were also measured at 24 hpi using human IFN-β specific ELISA kit. There was approximately five-fold increase in the levels of secreted IFN-β in HCMV infected-HFFs-NOD2 as compared to HCMV infected-HFFs-control (FIG. 4I). In non-infected HFFs-NOD2 the induction of IFN-β and IL8 was similar to that observed in control HFFs (FIG. 4H-J).

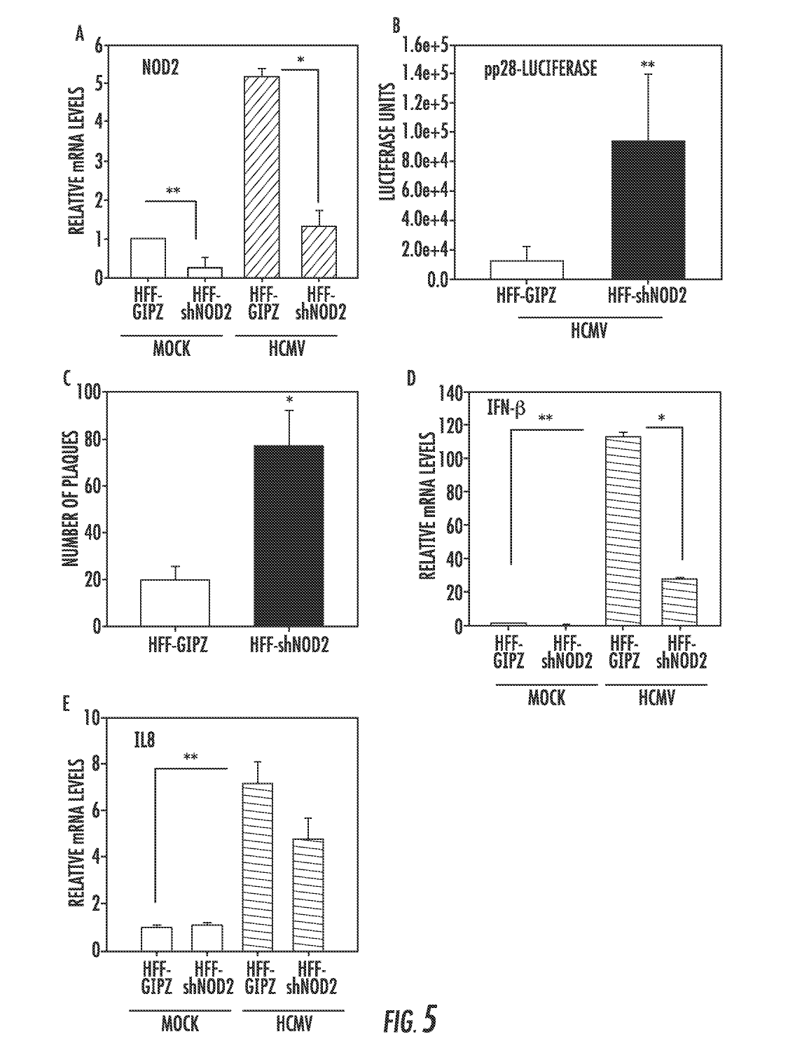

NOD2 knockdown (KD) results in enhanced HCMV replication and decreased levels of cytokines: A short hairpin (shRNA) pGIPZ lentivirus system was used to KD NOD2 expression in the two cell lines: HFFs and U373 cells. NOD2 mRNA levels were decreased by approximately 75% at 72 h in non-infected and HCMV-infected (MOI=1) NOD2-KD HFFs (HFF-shNOD2) compared to control shRNA (HFF-GIPZ) transduced cells (FIG. 5A). Supernatants from infected HFF-shNOD2 and HFF-GIPZ cells were collected at 96 hpi and used to infect fresh HFFs (second cycle). Luciferase activity, measured in the newly-infected cells at 72 hpi (a measure of infectious progeny released after single cycle infection), was increased by approximately 10-fold in HFF-shNOD2 cells compared to infected HFF-GIPZ control cells (FIG. 5B). Cell-free supernatants collected from HCMV infected-HFF-GIPZ and HFF-shNOD2 cells at 3 dpi were used for virus yield assay. There was approximately 4-fold increase in the number of plaques in cells infected with supernatants from HFF-shNOD2 cells as compared to the cells infected with supernatants from HFF-control cells (FIG. 5C). IFN-β mRNA levels measured in cell lysates were significantly reduced in HCMV infected HFF-shNOD2 cells as compared to infected HFF-GIPZ cells after 72 h (FIG. 5D). There was a modest decrease in IL8 transcripts in HCMV infected HFF-shNOD2 cells as compared to infected HFF-GIPZ cells after 72 h (FIG. 6E). NOD2-KD in U373 cells showed a similar pattern of enhanced virus replication and decreased cytokine responses to that observed in HFFs, suggesting this is an important mechanism for controlling HCMV replication.

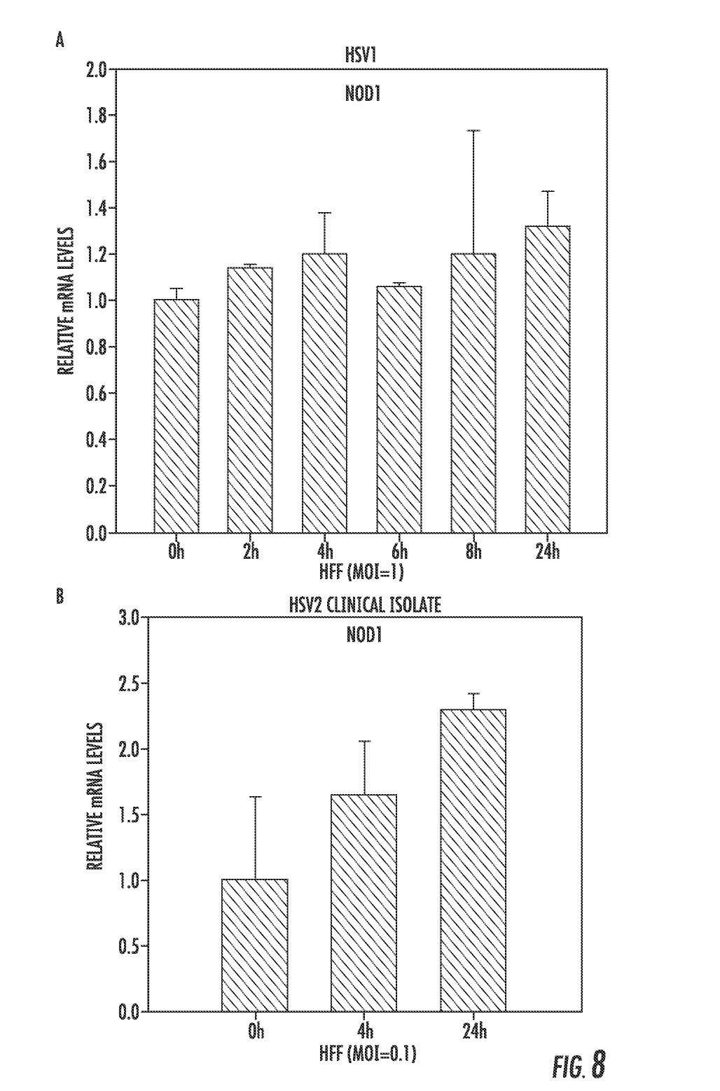

In NOD2-KD U373 cells (U373-shNOD2) NOD2 mRNA levels were decreased by approximately 80% at 72 h in both non-infected and HCMV-infected (MOI=1) cells (FIG. 9A) compared to control shRNA (U373-GIPZ) expressing cells. HCMV pp28-luciferase expression (measured at 96 hpi), HCMV DNA replication (measured at 48 hpi), and virus DNA yield in supernatants (measured at 96 hpi) from U373-shNOD2 cells and control U373-GIPZ revealed a significant increase in HCMV replication in the U373-shNOD2 cells (FIG. S2B): there was a 9-fold increase in virus DNA yield, 6-fold increase in DNA replication and 5-fold increase in luciferase activity. Quantification of mRNA expression of IFN-β and IL8 revealed a 3.5-fold decrease of IFN-β and 2-fold decrease in IL8 in U373-shNOD2 cells as compared to U373-GIPZ cells (FIGS. S2C, D).

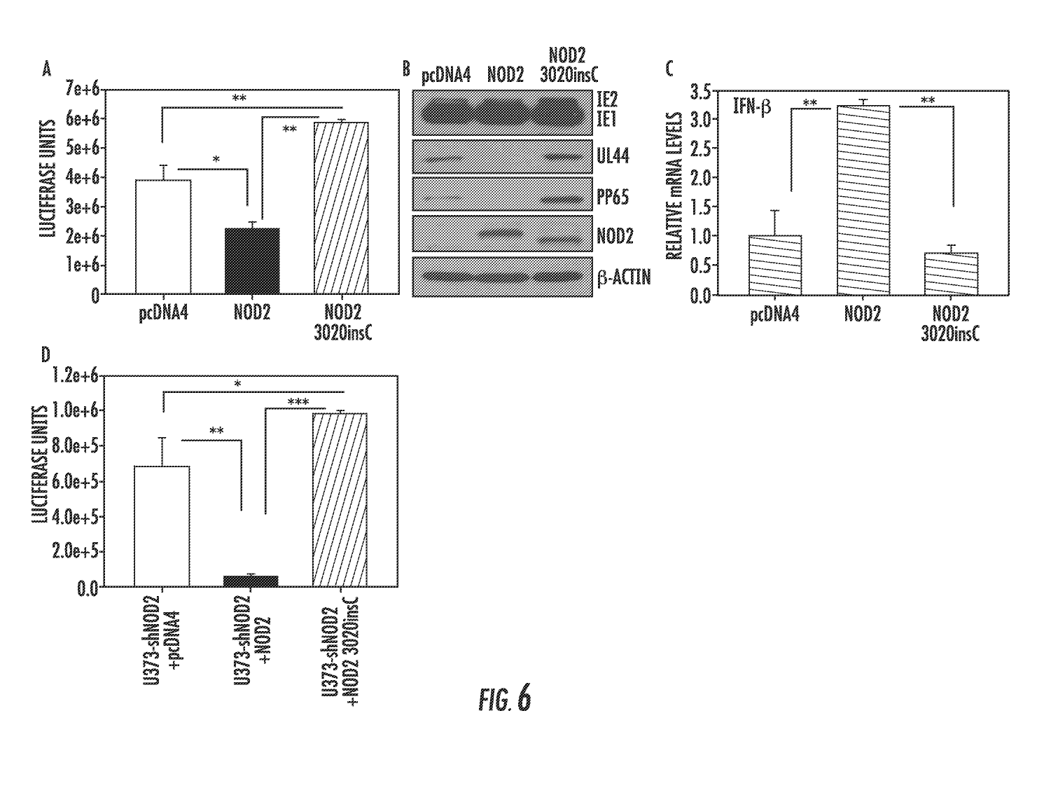

Overexpression of NOD2 mutant (3020insC) results in increased HCMV replication. The frameshift substitution at amino acid 1007 in the NOD2 gene stems from an insertion mutation resulting in a truncated NOD2 and impairing its ability to recognize microbial components. Patients with Crohn's disease that are homozygous for 3020insC demonstrate a much more severe disease phenotype [40]. Since NOD2 KD and its overexpression in HFFs and U373 showed similar effects on HCMV replication and antiviral responses we tested the effect of NOD2 3020insC mutant on HCMV replication in U373 cells. While overexpression of wild-type NOD2 resulted in significantly reduced HCMV replication, overexpression of the NOD2 mutant 3020insC in U373 resulted in increased virus replication (FIG. 7A). The expression of NOD2 3020insC was confirmed by western blot (FIG. 7B). IFN-β transcripts were measured in infected U373 cells transfected with pcDNA4, NOD2 or NOD2 3020insC mutant, demonstrating the IFN-β levels were not increased in cells transfected with the NOD2 3020insC, while induced upon transfection with the wild-type NOD2 (FIG. 7C).

NOD2 rescue in KD-cells restores the ability to restrict HCMV replication: U373 cells stably expressing control GIPZ shRNA (U373-GIPZ) or NOD2 shRNA (U373-shNOD2) were transiently transfected with either control plasmid (pcDNA4/HisMax) or a plasmid expressing NOD2-cDNA (pcDNA4/HisMax-NOD2). Twenty four hours following transfection, cells were infected with HCMV and luciferase activity was measured at 96 hpi. As shown in FIG. S2B, enhanced HCMV replication was observed in U373-shNOD2 cells as compared to virus replication in control U373-GIPZ cells, while overexpression of NOD2 resulted in significant decrease in HCMV replication. Transfection of the NOD2 gene into NOD2-KD U373 cells restored NOD2 function and resulted in restriction of HCMV replication, clearly demonstrating the specific role of NOD2 in HCMV replication (FIG. 7B). However, rescue of NOD2 3020insC in U373-shNOD2 did not lead to restricted HCMV replication (FIG. 7D).

NOD2 activates the NF-κB and the IFN pathway in HCMV-infected HFFs: Levels of NF-κB and phosphorylated forms of IRF3 were measured by western blot in cytoplasmic and nuclear extracts of HFF-shNOD2 cells and control HFF-GIPZ cells at 24 hpi. Compared to the control HFF-GIPZ cells, in which HCMV infection resulted in NF-κB localization to the nucleus, in the HFF-shNOD2 cells NF-κB remained in the cytoplasm (FIG. 7). Similarly, IRF3 was not activated in HFF-shNOD2 cells. While a phosphorylated form of IRF3 was observed in the nuclei extracted from HCMV-infected HFF-GIPZ cells, the level of nuclear IRF3 in HCMV-infected shNOD2 and its phosphorylated form were significantly decreased. These results suggest NOD2 serves as a central hub, activating both the NF-κB pathway and the IFN pathway following its induction in HCMV-infected cells.

### Discussion

We report for the first time that NOD2 is induced by HCMV and plays a significant role in restricting its replication. Infection of HFFs and U373 glioma cells with laboratory-adapted strains and a clinical isolate of HCMV resulted in a significant induction of NOD2 as early as 2 h after virus infection, required an intact viral genome and persisted throughout a full replication cycle. HSV1 and HSV2 did not induce NOD2 expression in infected HFFs. Overexpression of NOD2 resulted in decreased HCMV replication and enhanced antiviral and pro-inflammatory cytokine responses. NOD2 silencing (or transfection with the NOD2 mutant 3020insC) resulted in enhanced HCMV replication and decreased IFN-β levels. Reintroducing NOD2 into KD cells resulted in restriction of virus replication. Taken together, NOD2 plays a role in recognizing HCMV and restricting its replication. Although prior large scale transcriptomics studies did not report on NOD2 induction in HCMV-infected HFFs, RIPK2, a critical kinase downstream of NOD2 was significantly induced at 24 hpi [39].

Induction of NLRs result in activation of several signaling pathways: 1) The classic pathway is the NF-κB. Upon activation, NOD1/2 recruit RIPK2 [17] which promotes the K63-linked polyubiquitylation of the regulator NEMO/IKKγ and activation of the kinase transforming growth factor-β-activated kinase 1 (TAK1), which are prerequisites for the activation of the IKK complex. IKK activation results in degradation of the NF-κB inhibitor IκBα and the translocation of NF-κB to the nucleus, where transcription of NF-κB-dependent target genes occurs. RIPK2 is critical for NOD1- and NOD2-mediated NF-κB activation because NOD1 and NOD2 signaling is abolished in RIPK2-deficient cells [41]. In addition to the activation of the NF-κB pathway, NOD2 stimulation results in activation of the MAPKs p38, ERK and JNK [42]. 2) Alternative pathways which may or may not require RIPK2 include the induction of type I IFN and autophagy [24, 43-45] Inhibition of HCMV replication appears to involve its downstream kinase, RIPK2, because overexpression of RIPK2 resulted in decreased HCMV replication. Given our findings of changes in IFN-β levels as a result of NOD2 KD or overexpression, it is possible that RIPK2-induction by NOD2 activates both the classical and alternative pathways in HCMV-infected cells. HCMV infection was reported to activate IRF3, a process that required STING, an endoplasmic reticulum-resident protein involved in DNA sensing [14]. In the case of Helicobacter pylori, RIPK2 induction by NOD1, activated IKKε and IRF7, followed by the synthesis of type I IFN and signaling of the later through IFN-stimulated gene factor 3 (ISGF3) [24]. Mycobacterium tuberculosis activated NOD2-RIPK2, which stimulated the activity of IRF5 and induced transcription of IFNα/β [43]. NOD2 activation in HCMV-infected cells appears to induce NF-κB and IFN signaling pathways at least in part through RIPK2. Taken together, NOD2 may act as a central PRR, but the downstream signaling pathways are pathogen-determined and governed by specific virus and cellular components. Additional studies will determine more specifically the downstream signaling pathways activated by NOD2 in HCMV-infected cells as well as potential interaction between NOD2 and TLR2

Episodes of HCMV colitis have been reported in patients with inflammatory bowel disease (IBD), both ulcerative colitis and Crohn's disease [46-48] and were thought to result from virus reactivation in patients receiving immunosuppressive therapy. The fact that NOD2 is a susceptibility gene for Crohn's disease triggered our study for its potential role in HCMV recognition [20]. Since our results show that NOD2 mutation (3020insC) results in enhanced HCMV replication, it is possible that NOD2, a susceptibility gene for Crohns's disease, may influence susceptibility to HCMV infection. Although not tested here, based on our data, we suggest that HCMV colitis in patients with Crohn's disease could represent a specific outcome of virus-host interaction in a subset of patients that carry mutations in the NOD2 gene. Additional studies using epithelial cells and clinical material will be required to prove our hypothesis. Although NLRs have been traditionally thought to sense bacterial pathogens, our data suggest a wider role for NOD2 in sensing viruses including persistent DNA viruses such as HCMV. In vivo studies are needed to confirm these findings and to elucidate the role of HCMV in the intestinal microbiome, for which NOD2 is a key regulator linking it to mucosal immunity [49].

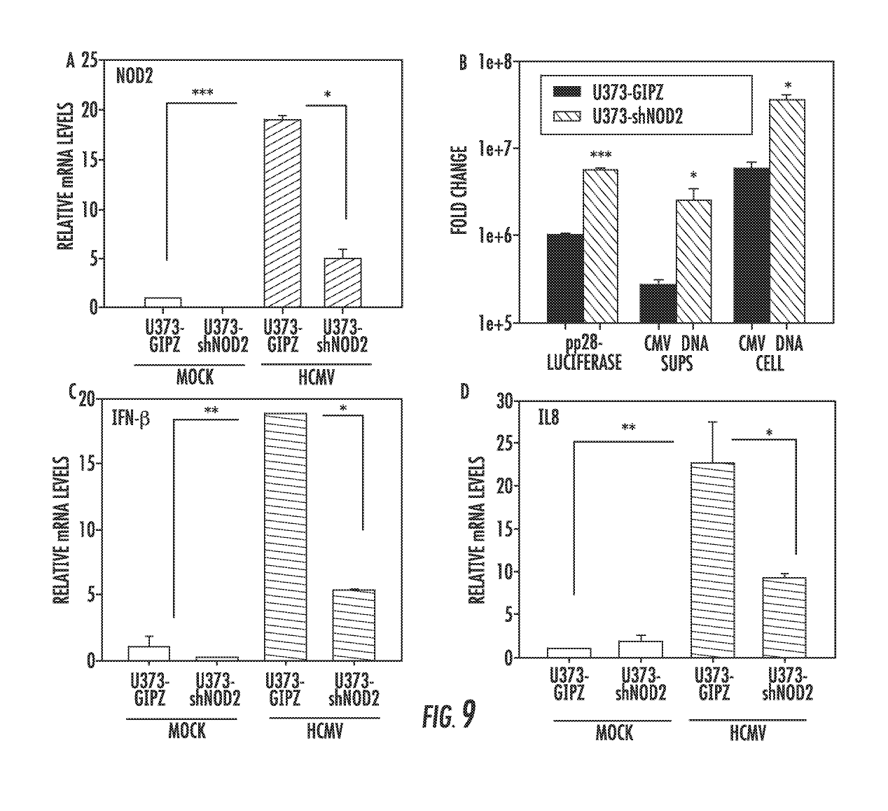

NOD1 recognizes CMV. Knockdown (KD) of NOD1 using lentivirus transduction results in enhanced CMV replication (FIG. 10A). Overexpression of NOD1 results in significant decrease in CMV replication (FIG. 10B). Virus replication was measured using the pp28-luciferase recombinant CMV strain which expresses luciferase under the control of the late pp28 CMV gene promoter. The GIPZ control shRNA and NOD1 shRNA were obtained from open biosystem. The clones with highest knockdown efficiency based on RNA and protein level were selected for these studies.

Pre-treatment of human fibroblasts with tri-DAP (NOD1 activator) followed by CMV infection resulted in significant reduction of plaque numbers (FIG. 11). These data suggest that NOD1 activation can limit CMV replication. It is likely that KD of NOD1 and NOD2 will result in synergistic effect on CMV replication. Similarly, overexpression of NOD1 and NOD1 may have synergistic activity in inhibiting CMV replication.

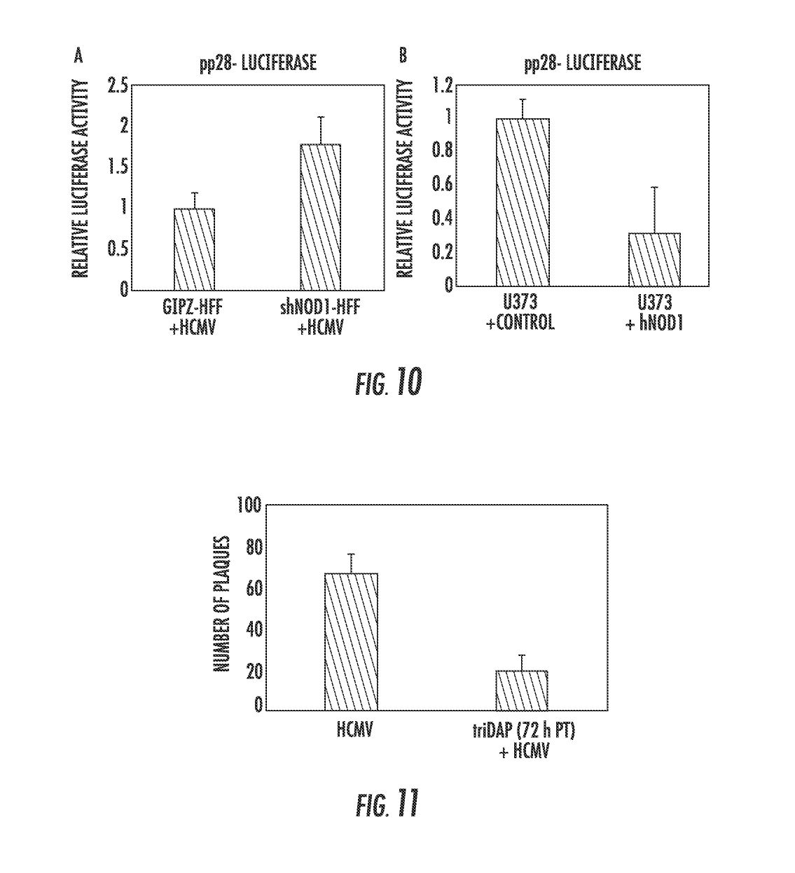

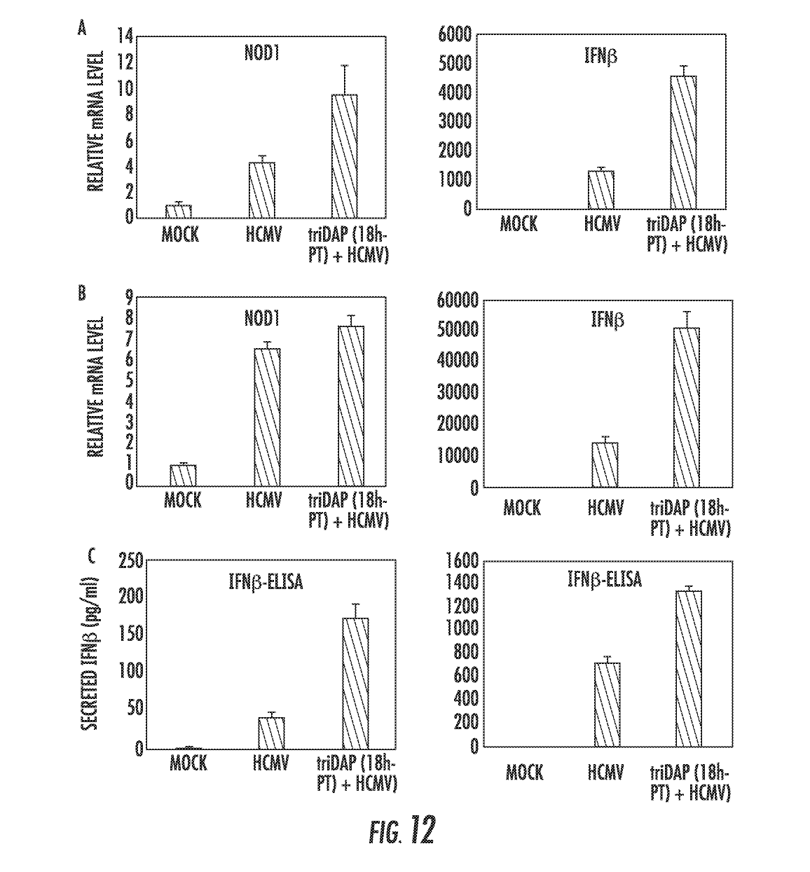

Pretreatment with triDAP resulted in enhanced interferon response (IFN-β), suggesting the effects of NOD1 on CMV replication involve the interferon pathway (FIG. 12). The activities of NOD1 and NOD2 were independent of each other, i.e., knockdown of NOD2 did not affect the expression of NOD1 or the NOD1-mediated IFN-β gene expression or NOD1 signaling pathway (FIG. 13).

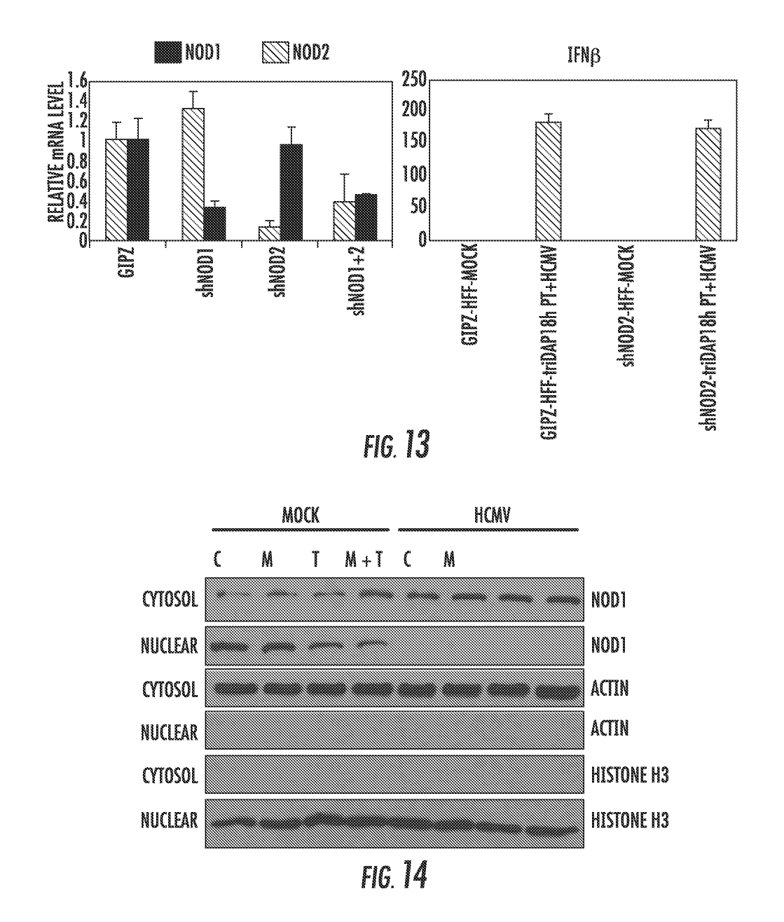

Intriguingly, CMV infection changes the localization of NOD1. We show for the first time that in non-infected HFFs NOD1 is localized to the nucleus while CMV infection causes its relocalization to the cytosolic compartment (FIG. 14).

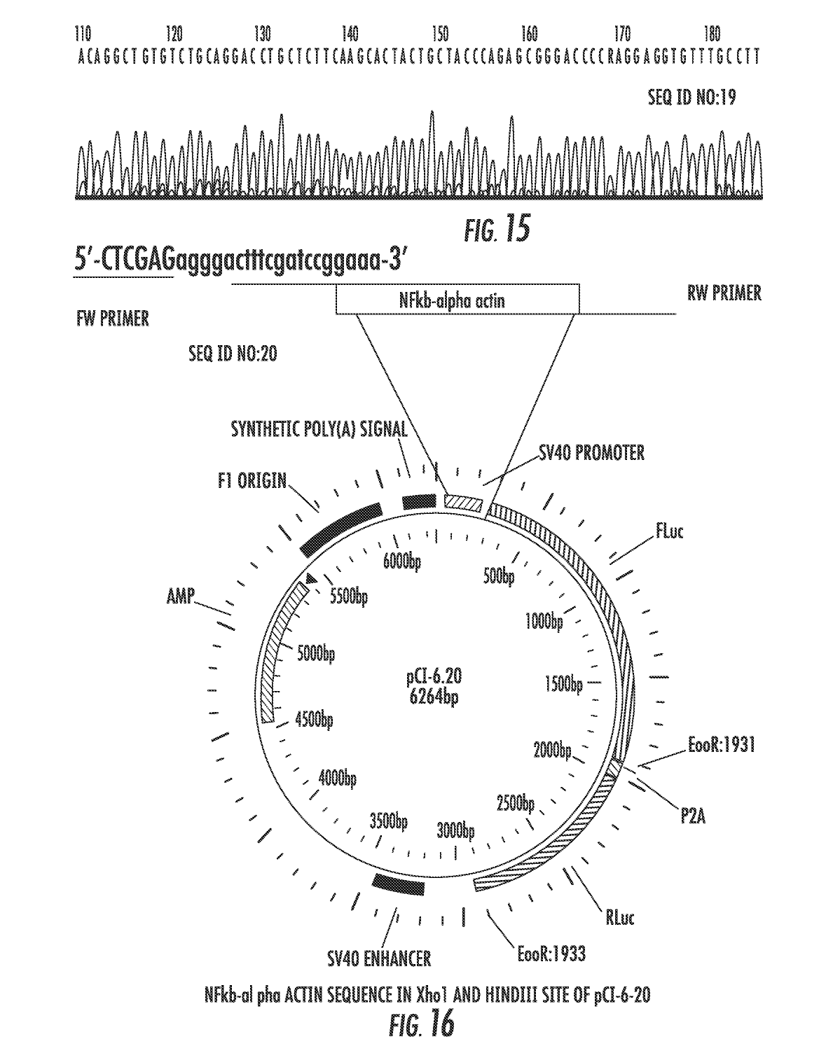

We recently treated a young toddler who was diagnosed with CMV colitis and viremia. His evaluation did not reveal any overt immunodeficiency. Based on our in vitro data, we hypothesized that his CMV disease may represent a specific host-pathogen interaction which could be explained by mutations in NOD1 and or NOD2. We sequenced specific regions of NOD1 and NOD2 from archived samples and found that the patient was harboring a mutation in NOD1 (E266K), as shown in FIG. 15, black line. One region of NOD2 (that includes the 3020C mutation associated with Crohn's disease) did not show any changes in this patient.

Our data show that NOD1 and NOD2 can serve as diagnostic markers for CMV and that polymorphisms in NOD1 (and potentially NOD2) can inform disease risk and be used as genetic markers. We suggest that polymorphisms in the NOD1 and NOD2 axis may also predict CMV disease. Genes that are part of this axis include RIPK2, XIAP, TAK1, TBK1/IKBKε, and IRF3/5/7. Mutations in all these genes can potentially be markers for CMV diseases. A table with some of the common mutations appears below.

TABLE 2
Markers for CMV

| Gene | Polymorphisms | Comments |
|---|---|---|
| NOD2 | R702W, G980R, | R334W is a gain of function |
| | L1007fs, R334W | mutation |
| NOD1 | E266K | |
| XIAP | E99X, G39C, K297T, | W323X-loss of RING domain, BIR2 |
| | W323X, C203Y | domain preserved. G39C, K297T- |
| | | NOD function preserved. |
| RIPK2 | K47A | |

We also suggest that modulation of NOD1 and NOD2 may provide a therapeutic platform for CMV. Towards this goal we used the plasmid pCL-6.20 which can be used for high throughput screen and replaced the SV40 promoter sequence with the NF-κB sequence (FIG. 16). This construct is currently used in the laboratory for optimization and measurement of specific activities through NOD1 and NOD2 and then will be applied to identify NOD1 and NOD2 activators using NIF.

### REFERENCES

1. Staras S A, Dollard S C, Radford K W, Flanders W D, Pass R F et al. (2006) Seroprevalence of cytomegalovirus infection in the United States, 1988-1994. Clin Infect Dis 43: 1143-1151.
2. Kenneson A, Cannon M J (2007) Review and meta-analysis of the epidemiology of congenital cytomegalovirus (CMV) infection. Rev Med Virol 17: 253-276.
3. Yow M D, Demmler G J (1992) Congenital cytomegalovirus disease—20 years is long enough. N Engl J Med 326: 702-703.
4. Griffiths P D, Clark D A, Emery V C (2000) Betaherpesviruses in transplant recipients. J Antimicrob Chemother 45 Suppl T3: 29-34.
5. Cooke G S, Hill A V (2001) Genetics of susceptibility to human infectious disease. Nat Rev Genet 2: 967-977.
6. Hill A V (2001) The genomics and genetics of human infectious disease susceptibility. Annu Rev Genomics Hum Genet 2: 373-400.
7. Molvig J, Baek L, Christensen P, Manogue K R, Vlassara H et al. (1988) Endotoxin-stimulated human monocyte secretion of interleukin 1, tumour necrosis factor alpha, and prostaglandin E2 shows stable interindividual differences. Scand J Immunol 27: 705-716.
8. Westendorp R G, Langermans J A, Huizinga T W, Elouali A H, Verweij C L et al. (1997) Genetic influence on cytokine production and fatal meningococcal disease. Lancet 349: 170-173.
9. Casanova J L, Abel L, Quintana-Murci L (2011) Human TLRs and IL-1Rs in host defense: natural insights from evolutionary, epidemiological, and clinical genetics. Annu Rev Immunol 29: 447-491.
10. Compton T, Kurt-Jones E A, Boehme K W, Belko J, Latz E et al. (2003) Human cytomegalovirus activates inflammatory cytokine responses via CD14 and Toll-like receptor 2. J Virol 77: 4588-4596.
11. Boehme K W, Guerrero M, Compton T (2006) Human cytomegalovirus envelope glycoproteins B and H are necessary for TLR2 activation in permissive cells. J Immunol 177: 7094-7102.
12. Kijpittayarit S, Eid A J, Brown R A, Paya C V, Razonable R R (2007) Relationship between Toll-like receptor 2 polymorphism and cytomegalovirus disease after liver transplantation. Clin Infect Dis 44: 1315-1320.
13. Paludan S R, Bowie A G, Horan K A, Fitzgerald K A (2011) Recognition of herpesviruses by the innate immune system. Nat Rev Immunol 11: 143-154.
14. DeFilippis V R, Alvarado D, Sali T, Rothenburg S, Fruh K (2010) Human cytomegalovirus induces the interferon response via the DNA sensor ZBP1. J Virol 84: 585-598.
15. Gariano G R, Dell'Oste V, Bronzini M, Gatti D, Luganini A et al. (2012) The intracellular DNA sensor IFI16 gene acts as restriction factor for human cytomegalovirus replication. PLoS Pathog 8: e1002498.
16. Kuenzel S, Till A, Winkler M, Hasler R, Lipinski S et al. (2010) The nucleotide-binding oligomerization domain-like receptor NLRC5 is involved in IFN-dependent antiviral immune responses. J Immunol 184: 1990-2000.
17. Kanneganti T D, Lamkanfi M, Nunez G (2007) Intracellular NOD-like receptors in host defense and disease. Immunity 27: 549-559.
18. Shaw M H, Reimer T, Kim Y G, Nunez G (2008) NOD-like receptors (NLRs): bona fide intracellular microbial sensors. Curr Opin Immunol 20: 377-382.
19. Shaw M H, Kamada N, Warner N, Kim Y G, Nunez G (2011) The ever-expanding function of NOD2: autophagy, viral recognition, and T cell activation. Trends Immunol 32: 73-79.
20. Ogura Y, Bonen D K, Inohara N, Nicolae D L, Chen F F et al. (2001) A frameshift mutation in NOD2 associated with susceptibility to Crohn's disease. Nature 411: 603-606.
21. Kobayashi K S, Chamaillard M, Ogura Y, Henegariu O, Inohara N et al. (2005) Nod2-dependent regulation of innate and adaptive immunity in the intestinal tract. Science 307: 731-734.
22. Hysi P, Kabesch M, Moffatt M F, Schedel M, Carr D et al. (2005) NOD1 variation, immunoglobulin E and asthma. Hum Mol Genet 14: 935-941.
23. Viala J, Chaput C, Boneca I G, Cardona A, Girardin S E et al. (2004) Nod1 responds to peptidoglycan delivered by the Helicobacter pylori cag pathogenicity island. Nat Immunol 5: 1166-1174.
24. Watanabe T, Asano N, Fichtner-Feigl S, Gorelick P L, Tsuji Y et al. (2010) NOD1 contributes to mouse host defense against Helicobacter pylori via induction of type I IFN and activation of the ISGF3 signaling pathway. J Clin Invest 120: 1645-1662.
25. Watanabe T, Asano N, Kitani A, Fuss I J, Chiba T et al. (2011) Activation of type I IFN signaling by NOD1 mediates mucosal host defense against Helicobacter pylori infection. Gut Microbes 2: 61-65.
26. Deshmukh H S, Hamburger J B, Ahn S H, McCafferty D G, Yang S R et al. (2009) Critical role of NOD2 in regulating the immune response to Staphylococcus aureus. Infect Immun 77: 1376-1382.
27. Shimada K, Chen S, Dempsey P W, Sorrentino R, Alsabeh R et al. (2009) The NOD/RIP2 pathway is essential for host defenses against Chlamydophila pneumoniae lung infection. PLoS Pathog 5: e1000379.
28. Opitz B, Puschel A, Schmeck B, Hocke A C, Rosseau S et al. (2004) Nucleotide-binding oligomerization domain proteins are innate immune receptors for internalized Streptococcus pneumoniae. J Biol Chem 279: 36426-36432.
29. Sabbah A, Chang T H, Harnack R, Frohlich V, Tominaga K et al. (2009) Activation of innate immune antiviral responses by Nod2. Nat Immunol 10: 1073-1080.
30. Lupfer C, Thomas P G, Anand P K, Vogel P, Milasta S et al. (2013) Receptor interacting protein kinase 2-mediated mitophagy regulates inflammasome activation during virus infection. Nat Immunol 14: 480-488.
31. Xu Y, Ahn J H, Cheng M, apRhys C M, Chiou C J et al. (2001) Proteasome-independent disruption of PML oncogenic domains (PODs), but not covalent modification by SUMO-1, is required for human cytomegalovirus immediate-early protein IE1 to inhibit PML-mediated transcriptional repression. J Virol 75: 10683-10695.
32. He R, Sandford G, Hayward G S, Burns W H, Posner G H et al. (2011) Recombinant Luciferase-Expressing Human Cytomegalovirus (CMV) for evaluation of CMV inhibitors. Virol J 8: 40.
33. Child S J, Jarrahian S, Harper V M, Geballe A P (2002) Complementation of vaccinia virus lacking the double-stranded RNA-binding protein gene E3L by human cytomegalovirus. J Virol 76: 4912-4918.
34. Kapoor A, He R, Venkatadri R, Forman M, Arav-Boger R (2013) Wnt modulating agents inhibit human cytomegalovirus replication. Antimicrob Agents Chemother 57: 2761-2767.
35. Tiscornia G, Singer O, Ikawa M, Verma T M (2003) A general method for gene knockdown in mice by using lentiviral vectors expressing small interfering RNA. Proc Natl Acad Sci USA 100: 1844-1848.
36. Tanaka Y, Kanda Y, Kami M, Mori S, Hamaki T et al. (2002) Monitoring cytomegalovirus infection by antigenemia assay and two distinct plasma real-time PCR methods after hematopoietic stem cell transplantation. Bone Marrow Transplant 30: 315-319.
37. Rosner M, Hengstschlager M (2008) Cytoplasmic and nuclear distribution of the protein complexes mTORC1 and mTORC2: rapamycin triggers dephosphorylation and delocalization of the mTORC2 components rictor and sin1. Hum Mol Genet 17: 2934-2948.
38. Girardin S E, Boneca I G, Viala J, Chamaillard M, Labigne A et al. (2003) Nod2 is a general sensor of peptidoglycan through muramyl dipeptide (MDP) detection. J Biol Chem 278: 8869-8872.
39. Browne E P, Wing B, Coleman D, Shenk T (2001) Altered cellular mRNA levels in human cytomegalovirus-infected fibroblasts: viral block to the accumulation of antiviral mRNAs. J Virol 75: 12319-12330.
40. Hugot J P, Chamaillard M, Zouali H, Lesage S, Cezard J P et al. (2001) Association of NOD2 leucine-rich repeat variants with susceptibility to Crohn's disease. Nature 411: 599-603.
41. Kobayashi K, Inohara N, Hernandez L D, Galan J E, Nunez G et al. (2002) RICK/Rip2/CARDIAK mediates signalling for receptors of the innate and adaptive immune systems. Nature 416: 194-199.
42. Hsu Y M, Zhang Y, You Y, Wang D, Li H et al. (2007) The adaptor protein CARD9 is required for innate immune responses to intracellular pathogens. Nat Immunol 8: 198-205.
43. Pandey A K, Yang Y, Jiang Z, Fortune S M, Coulombe F et al. (2009) NOD2, RIP2 and IRF5 play a critical role in the type I interferon response to Mycobacterium tuberculosis. PLoS Pathog 5: e1000500.
44. Homer C R, Richmond A L, Rebert N A, Achkar J P, McDonald C (2010) ATG16L1 and NOD2 interact in an autophagy-dependent antibacterial pathway implicated in Crohn's disease pathogenesis. Gastroenterology 139: 1630-41, 1641.
45. Travassos L H, Carneiro L A, Girardin S, Philpott D J (2010) Nod proteins link bacterial sensing and autophagy. Autophagy 6: 409-411.
46. Rahbar A, Bostrom L, Lagerstedt U, Magnusson I, Soderberg-Naucler C et al. (2003) Evidence of active cytomegalovirus infection and increased production of IL-6 in tissue specimens obtained from patients with inflammatory bowel diseases. Inflamm Bowel Dis 9: 154-161.
47. Kim J J, Simpson N, Klipfel N, Debose R, Barr N et al. (2010) Cytomegalovirus infection in patients with active inflammatory bowel disease. Dig Dis Sci 55: 1059-1065.
48. Matsuoka K, Iwao Y, Mori T, Sakuraba A, Yajima T et al. (2007) Cytomegalovirus is frequently reactivated and disappears without antiviral agents in ulcerative colitis patients. Am J Gastroenterol 102: 331-337.
49. Borm M E, van Bodegraven A A, Mulder C J, Kraal G, Bouma G (2008) The effect of NOD2 activation on TLR2-mediated cytokine responses is dependent on activation dose and NOD2 genotype. Genes Immun 9: 274-278.

1. A method for treating human cytomegalovirus (HCMV) in a patient in need thereof comprising administering an effective amount of a NOD1 pathway agonist and/or a NOD2 pathway agonist.

2. The method of claim 1, wherein the agonist is selected from the group consisting of a protein, a small molecule, an antibody, and an aptamer.

3. The method of claim 2, wherein the modulator is a small molecule.

4. The method of claim 1, wherein the NOD2 pathway agonist is muramyl dipeptide (MDP).

5. The method of claim 1, wherein the NOD1 pathway agonist is L-Ala-γ-D-Glu-mDAP (Tri-DAP).

6. A method for treating human cytomegalovirus (HCMV) in a patient in need thereof comprising administering an agent that increases the expression or activity of NOD1 and/or NOD2.

7. A method for identifying a subject as susceptible to or likely to develop a human cytomegalovirus infection comprising the steps of:
a. obtaining a biological sample from the subject;
b. performing an assay on the sample obtained from the subject to identify a mutation in NOD2; and
c. identifying the subject as susceptible to likely to develop human cytomegalovirus infection if the NOD2 mutation is identified.

8. The method of claim 7, wherein the assay of step (b) comprises sequencing of a region of the NOD2 gene comprising the mutation.

9. The method of claim 7, wherein the assay of step (b) comprises the steps of:
i. extracting DNA from the biological sample;
ii. contacting the DNA with a primer that specifically hybridizes to the NOD2 gene;
iii. amplifying by polymerase chain reaction (PCR) a region of the NOD2 gene that comprises the mutation; and
iv. sequencing the amplification product to identify the presence of the NOD2 mutation.

10. The method of claim 7, wherein the NOD2 mutation is 3020insC.

11. The method of claim 7, wherein the NOD2 mutation comprises R702W, G980R, L1007fs, and/or R334W.

12. The method of claim 7, further comprising performing an assay on the sample obtained from the subject to identify a mutation in one or more of vimentin, NOD1, OAS2, RIG-I, RIPK2, XIAP, Nemo (IKK gamma), IKK epsilon, IRF3, IRF5, and IRF7.

13. The method of claim 12, wherein the NOD mutation comprises E266K, the RIPK2 mutation comprises K47A, and the XIAP mutation comprises E99X, G39C, K297T, W323X, and/or C203Y.

14. The method of claim 7, further comprising the step of administering a treatment modality appropriate for a subject susceptible to or likely to develop human cytomegalovirus infection.

15. The method of claim 14, wherein the treatment modality for human cytomegalovirus infection comprises ganciclovir, valganciclovir, foscarnet, cidofovir, and/or cytomegalovirus immune globulin.

16. The method of claim 14, wherein the treatment modality comprises administering to the subject a NOD1 pathway agonist and/or a NOD2 pathway agonist.

17. A method for treating a subject having a human cytomegalovirus infection comprising the steps of:
d. obtaining a biological sample from the subject;
e. performing an assay on the sample obtained from the subject to identify a mutation in NOD1 or NOD2;
f. identifying the subject as susceptible to likely to develop human cytomegalovirus infection if the NOD1 and/or NOD2 mutation is identified; and
g. treating the subject with one or more treatment modalities appropriate for a subject having or likely to develop human cytomegalovirus infection.

18. The method of claim 17, wherein the assay of step (b) comprises sequencing of a region of the NOD1 or NOD2 gene comprising the mutation.

19. The method of claim 17, wherein the assay of step (b) comprises the steps of:
i. extracting DNA from the biological sample;
ii. contacting the DNA with primers that specifically hybridize to the NOD1 and NOD2 gene;
iii. amplifying by polymerase chain reaction (PCR) a region of the NOD1 and NOD2 gene that comprises the mutation; and
iv. sequencing the amplification product to identify the presence of the NOD1 and/or NOD2 mutation.

20. The method of claim 17, wherein the NOD2 mutation is 3020insC.

21. The method of claim 17, wherein the NOD2 mutation comprises R702W, G980R, L1007fs, and/or R334W.

22. The method of claim 17, further comprising performing an assay on the sample obtained from the subject to identify a mutation in one or more of vimentin, NOD1, OAS2, RIG-I, RIPK2, XIAP, Nemo (IKK gamma), IKK epsilon, IRF3, IRF5, and IRF7.

23. The method of claim 17, wherein the NOD mutation comprises E266K, the RIPK2 mutation comprises K47A, and the XIAP mutation comprises E99X, G39C, K297T, W323X, and/or C203Y.

24. The method of claim 17, wherein the treatment modality for human cytomegalovirus infection comprises ganciclovir, valganciclovir, foscarnet, cidofovir, and/or cytomegalovirus immune globulin.

25. The method of claim 17, wherein the treatment modality comprises administering to the subject a NOD1 and/or NOD2 pathway agonist.


SEQUENCE LISTING (20 sequences)

SEQ ID NO: 1 - DNA, 26 bp, Artificial Sequence
  NOD2 forward cDNA primer
ggatccatgggggaagagggtggttc

SEQ ID NO: 2 - DNA, 29 bp, Artificial Sequence
  NOD2 reverse cDNA primer
ctcgagtcaaagcaagagtctggtgtccc

SEQ ID NO: 3 - DNA, 30 bp, Artificial Sequence
  NOD2 forward cDNA primer
gggatccaccggtccaccatgggggaagag

SEQ ID NO: 4 - DNA, 37 bp, Artificial Sequence
  NOD2 reverse cDNA primer
gggatccacgcgttcattaagcgtagtctgggacgtc

SEQ ID NO: 5 - DNA, 27 bp, Artificial Sequence
  NOD1 forward primer
cctagacaacaacaatctcaacgacta

SEQ ID NO: 6 - DNA, 21 bp, Artificial Sequence
  NOD1 reverse primer
tttaccccaccgtcagtgatc

SEQ ID NO: 7 - DNA, 18 bp, Artificial Sequence
  NOD2 forward primer
gccacggtgaaagcgaat

SEQ ID NO: 8 - DNA, 21 bp, Artificial Sequence
  NOD2 reverse primer
ggaagcgagactgagcagaca

SEQ ID NO: 9 - DNA, 24 bp, Artificial Sequence
  IL8 forward primer
tgcagctctgtgtgaaggtgcagt

SEQ ID NO: 10 - DNA, 25 bp, Artificial Sequence
  IL8 reverse primer
cagtgtggtccactctcaatcactc

SEQ ID NO: 11 - DNA, 21 bp, Artificial Sequence
  IFNB forward primer
gattcatctagcactggctgg

SEQ ID NO: 12 - DNA, 20 bp, Artificial Sequence
  IFNB reverse primer
cttcaggtaatgcagaatcc

SEQ ID NO: 13 - DNA, 20 bp, Artificial Sequence
  ISG15 forward primer
gctccatgtcggtgtcagag

SEQ ID NO: 14 - DNA, 21 bp, Artificial Sequence
  ISG15 reverse primer
ctcgaaggtcagccagaacag

SEQ ID NO: 15 - DNA, 20 bp, Artificial Sequence
  GAPDH forward primer
ttggtatcgtggaaggactc

SEQ ID NO: 16 - DNA, 20 bp, Artificial Sequence
  GAPDH reverse primer
acagtcttctgggtggcagt

SEQ ID NO: 17 - DNA, 20 bp, Artificial Sequence
  Viperin forward primer
caagaggagaaagcagcagc

SEQ ID NO: 18 - DNA, 21 bp, Artificial Sequence
  Viperin reverse primer
caggagatagcgagaatgtcc

SEQ ID NO: 19 - DNA, 76 bp, Homo sapiens
acaggctgtgtctgcaggacctgctcttcaagcactactgctacccagagcgggaccccraggaggtgtttgcctt

SEQ ID NO: 20 - DNA, 26 bp, Artificial sequence
  Forward primer
ctcgagagggactttcgatccggaaa